(19) United States
(12) Patent Application Publication
(10) Pub. No.: US 2013/0184509 A1
(43) Pub. Date: Jul. 18, 2013

(54) Methods of Making Xylene Isomers

(71) Applicants: Hilary E. Schwartz, Milwaukee, WI (US); Jeffrey T. Miller, Naperville, IL (US); Brian J. Henley, Sandwich, IL (US); George A. Huff, Naperville, IL (US)

(72) Inventors: Hilary E. Schwartz, Milwaukee, WI (US); Jeffrey T. Miller, Naperville, IL (US); Brian J. Henley, Sandwich, IL (US); George A. Huff, Naperville, IL (US)

(73) Assignee: BP Corporation North America Inc., Houston, TX (US)

(21) Appl. No.: 13/783,654

(22) Filed: Mar. 4, 2013

Related U.S. Application Data
(63) Continuation of application No. 11/215,272, filed on Aug. 30, 2005, now abandoned.

(51) Int. Cl. C07C 6/00; C07C 5/27

(52) U.S. Cl. CPC C07C 6/00; C07C 5/2708
USPC 585/475

(57) ABSTRACT

Disclosed herein are methods of making xylene isomers. The methods generally include contacting an aromatics-comprising feed with a non-sulfided catalyst under conditions suitable for converting the feed to a product comprising xylene isomers. The catalyst includes a support impregnated with a hydrogenation component. The support includes a macroporous binder and a sieve selected from the group consisting of a medium pore sieve, a large pore sieve, and mixtures thereof. The selection of the sieve will depend upon the size of the molecules in the feed, intermediate, and product that can be expected from the catalytic reactions. When the molecules are expected to be large, a large pore sieve should be used. In contrast, when the molecules are expected to be smaller, either a large pore sieve, a medium pore sieve, or a mixture thereof may be used. The macropores within the support have been found to be especially beneficial because they help to overcome diffusional limitations observed when utilizing highly-active catalysts lacking such macropores.

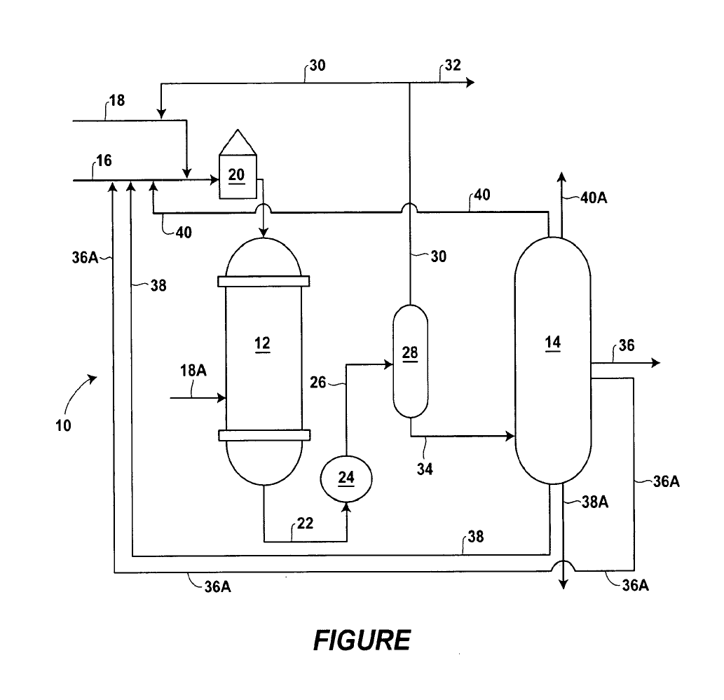

### BACKGROUND OF THE DISCLOSURE

1. Field of the Disclosure

The disclosure generally relates to methods of making xylene isomers and, more specifically, to methods of converting an aromatics-comprising feed to xylene isomers with the aid of a non-sulfided catalyst comprising a support impregnated with a hydrogenation component, wherein the support includes a macroporous binder and a sieve containing medium and/or large pores.

2. Brief Description of Related Technology

Hydrocarbon mixtures containing C₈ aromatics are often products of oil refinery processes including, but not limited to, catalytic reforming processes. These reformed hydrocarbon mixtures typically contain C₆₋₁₁ aromatics and paraffins, most of the aromatics of which are C₇₋₉ aromatics. These aromatics can be fractionated into their major groups, i.e., C₆, C₇, C₈, C₉, C₁₀, and C₁₁ aromatics. The C₈ aromatics fraction generally includes about 10 weight percent (wt. %) to about 30 wt. % non-aromatics, based on the total weight of the C₈ fraction. The balance of this fraction includes C₈ aromatics. Most commonly present among the C₈ aromatics are ethylbenzene (“EB”) and xylene isomers, including meta-xylene (“mX”), ortho-xylene (“oX”), and para-xylene (“pX”). Together, the xylene isomers and ethylbenzene are collectively referred to in the art and herein as “C₈ aromatics,” Typically, when present among the C₈ aromatics, ethylbenzene is present in a concentration of about 15 wt. % to about 20 wt. %, based on the total weight of the C₈ aromatics, with the balance (e.g., up to about 100 wt. %) being a mixture of xylene isomers. The three xylene isomers typically comprise the remainder of the C₈ aromatics, and are generally present at an equilibrium weight ratio of about 1:2:1 (oX:mX:pX). Thus, as used herein, the term “equilibrated mixture of xylene isomers” refers to a mixture containing the isomers in the weight ratio of about 1:2:1 (okmX:pX).

The product (or reformate) of a catalytic reforming process comprises C₆₋₁₂ aromatics (including benzene, toluene, and C₈ aromatics, which are collectively referred to as “BTX”). Byproducts of the process include hydrogen, light gas, paraffins, naphthenes, and heavy. C₉₊ aromatics. The BTX present in the reformate (especially toluene, ethylbenzene, and xylene) are known to be useful gasoline additives. However, due to environmental and health concerns, the maximum permissible level for certain aromatics (especially benzene) in gasoline has been greatly reduced. Nonetheless, the constituent parts of BTX can be separated in downstream unit operations for use in other capacities. Alternatively, benzene can be separated from the BTX and the resulting mixture of toluene and C₈ aromatics can be used as additives to boost the octane rating of gasoline, for example.

Benzene and xylenes (especially para-xylene) can be more marketable than toluene due to their usefulness in making other products. For example, benzene can be used to make styrene, cumene, and cyclohexane. Benzene also is useful in the manufacture of rubbers, lubricants, dyes, detergents, drugs, and pesticides. Among the C₈ aromatics, ethylbenzene generally is useful in making styrene when such ethylbenzene is a reaction product of ethylene and benzene. However, due to purity problems, the ethylbenzene that is present in the C₈ aromatics fraction cannot practically be used for styrene production. Meta-xylene is useful for making isophthalic acid, which itself is useful for making specialty polyester fibers, paints, and resins. Ortho-xylene is useful for making phthalic anhydride, which itself is useful for making phthalate-based plasticizers. Para-xylene is a raw material useful for making terephthalic acids and esters, which are used for making polymers, such as poly(butene terephthalate), poly(ethylene terephthalate), and poly(propylene terephthalate). While ethylbenzene, meta-xylene, and ortho-xylene are useful raw materials, demands for these chemicals and materials made therefrom are not as great as the demand for pare-xylene and the materials made from para-xylene.

In view of the higher values placed on benzene, C₈ aromatics, and products made therefrom, processes have been developed to dealkylate toluene to benzene, disproportionate toluene to benzene and C₈ aromatics, and transalkylate toluene and C₉₊ aromatics to C₈ aromatics. These processes are generally described in Kirk Othmer's “Encyclopedia of Chemical Technology,” 4^{th} Ed., Supplement Volume, pp. 831-863 (John Wiley & Sons, New York, 1998), the disclosure of which is incorporated herein by reference.

Specifically, toluene disproportionation (“TDP”) is a catalytic process wherein two moles of toluene are converted to one mole of xylene and one mole of benzene, such as:

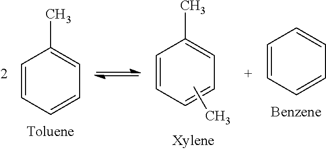

Other methyl disproportionation reactions include a catalytic process wherein two moles of a C₉ aromatic are converted to one mole of toluene and heavier hydrocarbon components (i.e., C₁₀₊ heavies), such as:

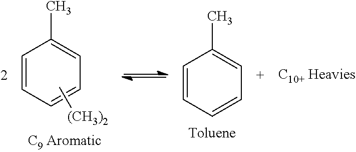

Toluene transalkylation is a reaction between one mole of toluene and one mole of a C₉ aromatic (or higher aromatic) to produce two moles of xylene, such as:

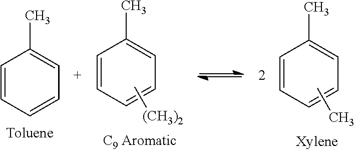

Other transalkylation reactions involving C₉ aromatics (or higher aromatics) include the reaction with benzene to produce toluene and xylene, such as:

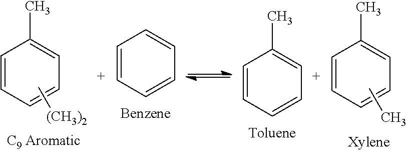

As shown in the foregoing reactions, the methyl and ethyl groups associated with the C₉ aromatics and xylene molecules are shown generically as such groups can be found bound to any available ring-forming carbon atoms to form the various isomeric configurations of the molecule. Mixtures of xylene isomers can be further separated into their constituent isomers in downstream processes. Once separated, the isomers can be further processed (e.g., isomerized, separated, and recycled) to obtain a substantially pure para-xylene, for example.

in theory and in view of the foregoing reactions, a mixture of C₉ aromatics can be converted to xylene isomers and/or benzene. Xylene isomers can be separated from benzene by fractional distillation, for example.

Heretofore, persons having ordinary skill in the art of disproportionation and transalkylation reactions would perform the above reactions with the aid of a catalyst depending upon which aromatic was ultimately sought. For example, U.S. Pat. Nos. 5,907,074; 5,866,741; 5,866,742; and, 5,804,059, each assigned to the Phillips Petroleum Company (“Phillips”), generally disclose disproportionation and transalkylation reactions wherein certain fluid feeds containing C₉₊ aromatics are converted to BTX. Though these patents state that the origin of the fluid feeds is not critical, each expresses a strong preference for fluid feeds derived from the heavies fraction of a product obtained by a hydrocarbon (particularly gasoline) aromatization reaction, which typically is carried out in a fluid catalytic cracking (“FCC”) unit. Low-value, liquid feeds comprising large (or long) hydrocarbons are vaporized in the FCC unit and, in the presence of a suitable catalyst, are cracked into lighter molecules capable of forming products that can be blended into higher-valued diesel fuel and high-octane gasoline. Byproducts of the FCC unit include a lower-valued, liquid heavies fraction, which constitutes the fluid feeds preferred according to the teachings of these patents. The very origin of the preferred fluid feeds, suggests that the feeds comprise sulfur-comprising compounds, paraffins, olefins, naphthenes, and polycyclic aromatics (“polyaromatics”).

According to the '074 patent, BTX are generally substantially absent from the feeds preferred therein and, therefore, no significant transalkylation of BTX occurs as a side reaction to the primary disproportionation and transalkylation reactions. The primary reactions described therein occur in the presence of a hydrogen-containing fluid and a catalyst comprising a metal oxide-promoted, Y-type zeolite having incorporated therein an activity modifier (i.e., oxides of sulfur, silicon, phosphorus, boron, magnesium, tin, titanium, zirconium, germanium, indium, lanthanum, cesium, and combinations of two or more thereof). The activity modifier helps to combat the deactivating effect (or poisoning effect) that sulfur-comprising compounds have on metal oxide impregnated catalysts.

According to the '741, '742, and '059 patents, BTX are generally substantially absent from the feeds preferred therein and, therefore, no significant transalkylation of BTX occurs as a side reaction to the primary disproportionation and transalkylation reactions. However, BTX can be present where alkylation of such chemicals by the C₉₊ aromatics is secondarily desired. According to the '741 patent, these primary and secondary reactions occur in the presence of a hydrogen-containing fluid and a catalyst comprising a beta-type zeolite having incorporated therein an activity promoter (e.g., molybdenum, lanthanum, and oxides thereof). According to the '742 patent, the primary and secondary reactions occur in the presence of a hydrogen-containing fluid and a catalyst comprising a beta-type zeolite having incorporated therein a metal carbide. According to the '059 patent, the primary and secondary reactions occur in the presence of a hydrogen-containing fluid and a catalyst comprising a metal oxide-promoted, mordenite-type zeolite.

The stated purpose underlying the teachings of each of the foregoing patents is to convert C₉₊ aromatics to BTX. Given this purpose, the patents disclose a specific combination of fluid feeds, catalysts, and reaction conditions suitable to obtain BTX. These patents do not, however, disclose or teach how to obtain any single BTX component (much less xylene isomers) to the minimization of the other BTX components. With respect to each of these, the presence of sulfur in the fluid feeds detrimentally converts the metal or metal oxide in the catalyst to a metal sulfide over time. Metal sulfides have a much lower hydrogenation activity than metal oxides and, therefore, the sulfur poisons the activity of the catalyst. Furthermore, the olefins, paraffins, and polyaromatics present in the feed rapidly deactivate the catalyst, and are converted to undesirable light gas.

In contrast to the foregoing patents, U.S. Patent Application Publication No. 2003/0181774 A1 (Kong et al.) discloses a transalkylation method of catalytically converting benzene and C₉₊ aromatics to toluene and C₈ aromatics. According to Kong et al., the method should be carried out in the presence of hydrogen in a gas-solid phase, fixed-bed reactor having a transalkylation catalyst comprising H-zeolite and molybdenum. The stated purpose behind Kong et al.'s method is to maximize production of toluene for subsequent use as a feed in a downstream selective disproportionation reactor, and to use the obtained C₈ aromatics byproduct as a feed in a downstream isomerization reactor. By selective disproportionation of the toluene to para-xylene. Kong et al. suggest how to ultimately convert a mixture of benzene and C₉₊ aromatics to para-xylene. However, such a suggestion disadvantageously requires multiple reaction vessels (e.g., a transalkylation reactor, and a disproportionation reactor) and, importantly, does not teach how to maximize the amount of xylene isomers produced from the transalkylation reaction, while concomitantly minimizing the production of toluene and ethylbenzene.

U.S. Patent Application Publication No. 2003/0130549 A1 (Xie et al.) discloses a method of selectively disproportionating toluene to obtain benzene and a xylene isomers stream rich in para-xylene, and transalkylating a mixture of toluene and C₉₊ aromatics to obtain benzene and xylene isomers. According to Xie et al., the different reactions are carried out in the presence of hydrogen in separate reactors each containing a suitable catalyst (i.e., a ZSM-5 catalyst for the selective disproportionation and a mordenite. MCM-22 or beta-zeolite for the transalkylation). Downstream processing is used to obtain para-xylene from the produced xylene isomers. The method disclosed by Xie et al. suggests that large volumes of benzene and ethylbenzene are desirably produced. Xie et al., however, do not suggest how to maximize the amount of xylene isomers produced from the transalkylation reaction, while concurrently minimizing the production of benzene and ethylbenzene.

U.S. Patent Application Publication No. 2001/0014645 A1 (Ishikawa et al.) discloses a method of disproportionating C₉₊ aromatics into toluene, and transalkylating C₉₊ aromatics and benzene to toluene and C₈ aromatics for use as gasoline additives. The use of benzene as a reactant in the transalkylation reaction suggests an attempt by Ishikawa et al. to rid low-value gasoline fractions of benzene. Given the stated use and suggestion to rid gasoline of benzene, one skilled in the art would desire ethylbenzene in the C₈ aromatics to maximize gasoline yields. Moreover, the skilled artisan will take precautions to ensure that the produced ethylbenzene is not unintentionally cracked to a benzene—which is sought to be removed from gasoline fractions. The disclosed reactions are carried out in the presence of hydrogen and a large-pore zeolite impregnated with a Group VIB metal and preferably sulfided. Generally, portions of the benzene and C₊ aromatics are converted to a product stream mostly comprising BTX. From the BTX product stream, benzene is removed and recycled back to the feed. Ultimately, toluene and C₈ aromatics are obtained from the benzene/C₉₊ aromatics feed. The transalkylating reaction is carried out with a large molar excess of benzene to C₉₊ aromatics (i.e., between 5:1 to 20:1) to obtain toluene and C₈ aromatics (including ethylbenzene). Ishikawa et al., however, do not suggest how to maximize the amount of xylene isomers produced in the transalkylation reaction, while also minimizing the production of toluene, benzenes, and C₁₀ aromatics.

The foregoing publications do not disclose and do not teach or suggest to a person having ordinary skill in the art how to maximize the production of xylene isomers from an aromatics-comprising feed, while minimizing the production of the other BTX components, non-aromatics, and heavies. Moreover, the prior art does not disclose and does not teach or suggest to the skilled artisan a highly active catalyst suitable to convert an aromatics-comprising feed to xylene isomers. The catalyst disclosed in each of the foregoing publications is specially selected to convert a specific feed to a specific end-product. There are many competing considerations when designing a catalyst suitable for converting a specific feed to a specific end-product. Among those considerations are the desired activity, (shape) selectivity, and diffusional limitations that result from the activity and selectivity. A highly active catalyst is desirable to maximize conversion of the feed, and selectivity is desirable to obtain a product containing certain molecules to the minimization of other molecules, and to purify the molecules comprising the product of the conversion (i.e., to destroy or separate undesired molecules in the product from the specific molecules that will diffuse through the catalyst). The conversion often includes byproducts undesired for a variety of reasons. For example, certain byproducts can be highly reactive and can undesirably react with and convert the desired product into other (less desired) molecules.

International (PCT) Publication WO 04/056475 generally discloses a catalytic conversion of ethylene and benzene to ethylbenzene and undesired by products, such as low molecular weight products (e.g., ethylene), biphenyl ethanes, and polyethyl benzenes. When ethyl groups (and higher alkyl groups) are removed from aromatic compounds they exist as ethylene groups (and higher alkylene groups), which are highly reactive and form the undesired byproducts. For example, free ethylene groups in the mixture will re-react with other portions of the benzene to yield biphenyl ethanes and polyethyl benzenes. The yield of these undesired byproducts is, according to the '475 publication, minimized with a specially-designed catalyst that includes a support formed from a large pore zeolite and an inorganic binder. The support is formed with the aid of a pore former to include mesopores and macropores, and has a pore volume of at least 0.7 cubic centimeters per gram. The larger pores and pore volume are stated therein to improve the diffusion characteristics of the catalyst. The improved diffusion provides faster throughput of the reactants and a shorter residence time, which, in turn, lead to a lower likelihood and diminished ability for the highly reactive ethylene to form the undesired byproducts. Moreover, large pores and pore volume also are stated therein to improve the diffusivity of the large polyethylated aromatic molecules that are present in these reactions.

The diffusional limitations addressed in the '475 publication are, of course, peculiar to the particular conversion described therein. Even if a highly-active catalyst was available to convert an aromatics-comprising feed to xylene isomers, those peculiar diffusional limitations would not be expected with such a conversion. Moreover, the use of a large pore support or a pore volume of the type disclosed in the '475 publication would not be expected to assist de-methylation, methyl-disproportionation and methyl-transalkylation reactions because methyl groups are not nearly as reactive as olefins (e.g., ethylene), do not typically exist as a gas in these reactions, and do not re-react with BTX and C₉₊ aromatics in the same way that ethylene and higher alkylenes react. Methyl groups are chemically slow reactants and, therefore, would not be expected by those having ordinary skill in the art to present the diffusion concerns that olefins present. Indeed, because the de-methylation, methyl-disproportionation, and methyl-transalkylation reactions are slow relative to the rate at which the molecules diffuse, the skilled artisan would not consider a catalyst support with such high pore volume and such large pores to be particularly beneficial for these reactions.

The prior art does not disclose and does not teach or suggest to the skilled artisan a highly active catalyst suitable to convert an aromatics-comprising feed to xylene isomers. Nor does the prior art disclose, teach, or suggest the reaction conditions under which such a catalytic conversion should be performed to maximize the yield of xylene isomers. Absent such disclosure and teachings, the prior art, not surprisingly, recognizes no meaningful diffusional constraints affiliated with the conversion of aromatics-comprising feeds to xylene isomers.

### SUMMARY OF THE DISCLOSURE

It has now been discovered that the use of a bi-functional catalyst containing macropores provides surprising benefits in terms of, for example, improved activity (improved conversion of aromatics-comprising feed) without compromising the selectivity for xylene isomers, improved catalyst stability, the ability to convert feeds containing some non-aromatics and C₁₀₊ aromatics without deactivation of the catalyst, the ability to manufacture highly pure benzene, improved yield of xylene recovery in downstream para-xylene processing units, and the unexpected flexibility to accommodate multiple feed operations utilizing the same general process configuration and removing selected products of the conversion as desired.

Accordingly, disclosed herein are methods of making xylene isomers utilizing such catalysts. In one embodiment, the method includes contacting a feed comprising C₉ aromatics with a non-sulfided catalyst under conditions suitable for converting the feed to a product comprising xylene isomers. The catalyst includes a support impregnated with a hydrogenation component, and the support includes a macroporous binder and a large pore sieve.

In another embodiment, the method includes contacting a feed comprising C₆-C₈ aromatics and substantially free of C₉₊ aromatics with a non-sulfided catalyst under conditions suitable for converting the feed to a product comprising xylene isomers. The catalyst includes a support impregnated with a hydrogenation component, and the support includes a macroporous binder and a sieve selected from the group consisting of a medium pore sieve, a large pore sieve, and mixtures thereof.

Additional features may become apparent to those skilled in the art from a review of the following detailed description, taken in conjunction with the drawing, the examples, and the appended claims.

### BRIEF DESCRIPTION OF THE DRAWING FIGURE

For a more complete understanding of the disclosure, reference should be made to the following detailed description and the sole drawing FIGURE generally illustrating a flow diagram for a process suitable for performing the disclosed methods and embodiments thereof. While the disclosed methods are susceptible of embodiments in various forms, there are illustrated in the drawing (and will hereafter be described) specific embodiments of the methods, with the understanding that the disclosure is intended to be illustrative, and is not intended to limit the scope of the methods to the specific embodiments described and illustrated herein.

### DETAILED DESCRIPTION OF THE DISCLOSURE

The disclosure generally relates to methods of making xylene isomers, which are especially suitable as a chemical feedstock for the production of para-xylene. Our co-pending, commonly-assigned application Ser. No. 10/794,932 filed Mar. 4, 2004, the disclosure of which is incorporated herein by reference, describes the great benefits in the conversion of an aromatics-comprising feed to xylene isomers with the use of a non-sulfided catalyst containing a hydrogenation component. It has now been discovered that the full benefits of that conversion may not be practically realizable on a commercial production scale because of diffusional limitations encountered on that scale, especially when the catalyst is in the form of an extruded pellet/particle. For example, a support impregnated with a hydrogenation component, such as a Group VIB metal oxide, wherein the support is made up of a binder and a large pore sieve, has been found to be so active with respect to converting feeds containing C₉ aromatics, benzene, toluene, or mixtures thereof to a product containing xylene isomers, that significant portions of the active sites within the extruded form of the catalyst are under-utilized or not utilized at all. This is a diffusional limitation and is undesirable because significant volumes of a commercial production-scale reaction vessel will unnecessary be occupied by a catalyst (lacking macropores) whose active sites may not be utilized (or will be under-utilized).

This diffusional limitation can be addressed by decreasing the feed rate into the reaction vessel (to match the conversion rate); however, this will likely reduce the productivity of the method. Alternatively, the limitation can be addressed by maintaining the feed rate, but increasing the reactor volume and the amount of catalyst packed into that volume (to match the conversion rate); however, this will likely increase capital costs.

Quite unexpectedly, we have discovered that the diffusional limitation can be addressed by utilizing a catalyst (support) that contains macropores. With a macroporous catalyst, the feed can be introduced into the reactor and diffuse via the macropores to active sites in the sieve previously under-utilized (or not utilized at all). The presence of macropores in the catalyst effectively increases the rate of catalytic conversion to better match the residence time of the feed and converted product within the reactor. Thus, we have discovered that, while an extruded form of the metal-impregnated catalyst support lacking macropores can convert an aromatics-containing feed to xylene isomers, the extruded form of the catalyst containing macropores performs the conversion far more efficiently on a commercial production scale based on a given reactor volume. Furthermore, reactor volume need not be increased as a result of increased feed rates to achieve such efficient conversions.

Utilizing a catalyst containing macropores is counterintuitive because the molecules in the feed, recycle, and product are typically small enough to diffuse through the pores of a catalyst lacking macropores. Consequently, a person having ordinary skill in the art would not consider utilizing a catalyst containing macropores to perform the conversion. Moreover, absent the presence of the hydrogenation component, the rate of catalytic conversion would not exceed the rate at which the feed passes through the catalyst. Consequently, all of the active sites on the catalyst would be accessible by the feed (i.e., there would no (or few) under-utilized sites). Thus, we have now discovered that the great benefits of a highly-active, bifunctional catalyst can be better and more practically realized on a commercial scale if the extruded form of the catalyst (support) includes macropores.

As described in more detail below, xylene isomers can be obtained from various feeds that can include C₉ aromatics, toluene, benzene, and mixtures thereof. Some reactions with these feeds such as, for example, toluene disproportionation and toluene transalkylation, have been described above. Generally, the methods disclosed herein include contacting the feed with a catalyst under conditions suitable to convert the feed into a product that includes xylene isomers.

The catalyst includes a support impregnated with a hydrogenation component. The support includes a macroporous binder and a sieve selected from the group consisting of a medium pore sieve, a large pore sieve, and mixtures thereof. The selection of the sieve is based on the composition of the feed. For example, large pore sieves should be used for feeds containing C₉ aromatics, whereas medium pore sieves, large pore sieves, or mixtures of such sieves can be used for aromatics-comprising feeds containing only molecules smaller in size than C₉ aromatics or aromatics-comprising feeds substantially free of molecules having a size equal to or greater than C₉ aromatics. Although the disclosed methods ultimately seek to obtain xylene isomers, it is readily understood that competing reactions will produce byproduct aromatics (e.g., aromatics other than xylene isomers such as benzene) in addition to the desired xylene isomers. These byproducts may have beneficial value, however, the amount of these byproducts preferably is minimized. Consequently, in certain embodiments, the “product” can be more accurately considered an “intermediate” because it also contains byproduct aromatics. The methods, therefore, also can include separating at least a portion of the xylene isomers from the intermediate to produce a xylene-isomers lean intermediate and recycling the same to the feed.

Suitable feeds for use in accordance with the disclosed methods include those ultimately obtained from crude oil refining processes. Generally, crude oil is desalted and thereafter distilled into various components. The desalting step generally removes metals and suspended solids that could cause catalyst deactivation in downstream processes. The product obtained from the desalting step subsequently undergoes atmospheric or vacuum distillation. Among the fractions obtained via atmospheric distillation are crude or virgin naphtha, kerosene, middle distillates, gas oils and lube distillates, and heavy bottoms, which often are further distilled via vacuum distillation methods. Many of these fractions can be sold as finished products or can be further processed in downstream unit operations capable of changing the molecular structure of the hydrocarbon molecules either by breaking them into smaller molecules, combining them to form a larger more highly-valued molecule, or reshaping them into more highly-valued molecules. For example, crude or virgin naphtha obtained from the distillation step can be passed with hydrogen through a hydrotreating unit, which converts any residual olefins to paraffins, and removes impurities such as sulfur, nitrogen, oxygen, halides, heteroatoms, and metal impurities that can deactivate downstream catalysts. Exiting the hydrotreating unit is a treated naphtha lean or substantially free of impurities, a hydrogen-rich gas, and streams containing hydrogen sulfide and ammonia. The light hydrocarbons are sent to a reforming step (a “reformer”) to convert those hydrocarbons (e.g., nonaromatics) into hydrocarbons having better gasoline properties (e.g., aromatics). The treated naphtha, generally comprises aromatics (typically in the boiling range of C₆₋₁₀ aromatics), can serve as a feed suitable for conversion in accordance with the disclosed inventive methods.

Alternatively, a hydrocracking unit can take a feed comprising middle distillates and/or gas oil and convert that feed to light hydrocarbons having poor gasoline properties (i.e., naphtha) and little to no sulfur or olefins. The light hydrocarbons are then sent to a reformer to convert those hydrocarbons into hydrocarbons having better gasoline properties (e.g., aromatics).

Exiting the reformer is a reformate that is substantially free of sulfur and olefins and includes not only aromatics (typically in the boiling range of C₆₋₁₀ aromatics) but also paraffins and polyaromatics. Thus, in a subsequent step, paraffins and polyaromatics are removed to yield a product stream containing C₉ aromatics. Such a product stream can serve as a feed suitable for conversion in accordance with the disclosed inventive methods.

The composition of crude oil can vary significantly depending upon its source. Moreover, feeds suitable for use in accordance with the inventive methods disclosed herein are typically obtained as products of a variety of upstream unit operations and, of course, can vary depending upon the reactants/materials supplied to those unit operations. Oftentimes, the origin of those reactants/materials will dictate the composition of the feed obtained as a product of the unit operations. As described in more detail below, there are generally two types of feeds, which the inventive methods can convert to xylene isomers: those containing C₉ aromatics and those containing benzene and/or toluene that are substantially free of C₉ aromatics and molecules larger in size than C₉ aromatics.

As used herein, the term “aromatic” defines a major group of unsaturated cyclic hydrocarbons containing one or more rings, typified by benzene. See generally, “Hawley's Condensed Chemical Dictionary,” at p. 92 (13^{th} Ed., 1997). Generally a Cₙ aromatic refers to an aromatic compound having n carbon atoms. Furthermore, a Cₙ₊ aromatic refers to an aromatic compound having at least n carbon atoms. Thus, as used herein, the term “C₉ aromatics” means a mixture that includes any aromatic compound having nine carbon atoms. Preferably, the C₉ aromatics include 1,2,4-trimethylbenzene (pseudocumene), 1,2,3-trimethylbenzene (hemimellitene), 1,3,5-trimethylbenzene (mesitylene), meta-methylethylbenzene, ortho-methylethylbenzene, pare-methylethylbenzene, iso-propylbenzene, and n-propylbenzene. As used herein, “C₉₊ aromatics” means a mixture that includes any aromatic compound having at least nine carbon atoms, such as, for example a C₁₀ aromatic. Similarly, “C₁₀₊ aromatics” means a mixture that includes any aromatic compound having at least ten carbon atoms.

Along with feeds comprising C₉ aromatics, the feed typically will include numerous other hydrocarbons, many of which are only present in trace amounts. For example, the feed preferably is substantially free of non-aromatics such as, for example, paraffins and olefins. A feed that is substantially free of non-aromatics preferably comprises less than about 5 wt. % non-aromatics, and more preferably less than about 3 wt. % non-aromatics, based on the total weight of the feed. Although suitable feeds are preferably substantially free of non-aromatics, feeds containing non-aromatics can be processed by the disclosed methods as demonstrated in the examples reported below.

The feed should be substantially free of sulfur (e.g., elemental sulfur and sulfur-containing hydrocarbons and non-hydrocarbons). A feed that is substantially free of sulfur preferably comprises less than about 1 wt. % sulfur, more preferably less than about 0.1 wt. % sulfur, and even more preferably less than about 0.01 wt. % sulfur, based on the total weight of the feed.

In various preferred embodiments, the feed is substantially free of xylene isomers, toluene, ethylbenzene, and/or benzene. A feed that is substantially free of xylene isomers preferably comprises less than about 3 wt. % xylene isomers, and more preferably less than about 1 wt. % xylene isomers, based on the total weight of the feed. A feed that is substantially free of toluene preferably comprises less than about 5 wt. % toluene, and more preferably less than about 3 wt. % toluene, based on the total weight of the feed. A feed that is substantially free of ethylbenzene preferably comprises less than about 5 wt. % of ethylbenzene, and more preferably less than about 3 wt. % ethylbenzene, based on the total weight of the feed.

In other embodiments, however, the feed can include significant amounts of one or both of toluene and benzene. For example, in certain embodiments, the feed can include up to about 50 wt. % toluene, based on the total weight of the feed. Preferably, however, the feed includes less than about 50 wt. % toluene, more preferably less than about 40 wt. % toluene, even more preferably less than about 30 wt. % toluene, and most preferably less than about 20 wt. % toluene, based on the total weight of the feed. Similarly, in certain embodiments, the feed can include up to about 30 wt. % benzene, based on the total weight of the feed. Preferably, however, the feed includes less than about 30 wt. % benzene, and more preferably, less than about 20 wt. % benzene, based on the total weight of the feed.

Still further, in various embodiments, the feed can be substantially free of C₁₀₊ aromatics. The feed, however, need not be substantially free of C₁₀₊ aromatics. Generally, C₁₀₊ aromatics (“A₁₀₊”) will include benzenes having one or more hydrocarbon functional groups which, in the aggregate, have four or more carbons. Examples of such C₁₀₊ aromatics include, but are not limited to, C₁₀ aromatics (“A₁₀”), such as butylbenzene, (including isobutylbenzene and tertiarybutylbenzene), diethylbenzene, methylpropylbenzene, dimethylethylbenzene, tetramethylbenzene, and C₁₁ aromatics, such as trimethylethylbenzene, and ethylpropylbenzene, for example. Examples of C₁₀₊ aromatics also can include naphthalene, and methylnaphthalene. A feed that is substantially free of C₁₀₊ aromatics preferably comprises less than about 5 wt. % C₁₀₊ aromatics, and more preferably less than about 3 wt. % C₁₀₊ aromatics, based on the total weight of the feed.

As used herein the term “C₈ aromatics” means a mixture containing predominantly xylene isomers and ethylbenzene. In contrast, the term “xylene isomers,” as used herein, means a mixture containing meta-, ortho-, and para-xylenes, wherein the mixture is substantially free of ethylbenzene. Preferably, such a mixture contains less than three weight percent ethylbenzene based on the combined weight of the xylene isomers and any ethylbenzene. More preferably, however, such a mixture contains less than about one weight percent ethylbenzene.

A second type of feed that can be converted to xylene isomers by the inventive method is one that contains molecules (e.g. toluene) smaller than the C₉₊ aromatics discussed above, i.e., aromatics-comprising feeds substantially free of molecules having a size equal to or greater than C₉₊ aromatics. Generally, the feed will be rich in toluene and, thus, contain at least about 90 wt. % toluene, preferably about 95 wt. % toluene, and more preferably about 97 wt. % toluene, based on the total weight of the feed. This feed generally is convertible to xylene isomers via toluene disproportionation with catalytic sieves having smaller pores than the catalytic sieves necessary to convert feeds containing the C₉₊ aromatics discussed above. Generally, a feed that is substantially free of C₉ aromatics preferably comprises less than about 5 wt. % C₉ aromatics, more preferably less than about 3 wt. % C₉ aromatics, and more highly preferably less than about 1 wt. % C₉ aromatics, based on the total weight of the feed. This feed also should be substantially free of C₁₀₊ aromatics. A feed that is substantially free of C₁₀₊ aromatics preferably comprises less than about 5 wt. % C₁₀₊ aromatics, more preferably less than about 3 wt. % C₁₀₊ aromatics, and more highly preferably less than about 1 wt. % C₁₀₊ aromatics, based on the total weight of the feed. The presence of C₉ aromatics and C₁₀₊ aromatics in the feed would limit the ability to use smaller pore catalytic sieves because these molecules will not be able to pass through the sieve and will eventually clog the same rendering the catalytic material less useful or even useless. Consequently, to advantageously utilize smaller pore catalytic sieves (e.g., medium pore sieves), the feed should be substantially free of these larger molecules.

As with the C₉ aromatics comprising feed, a feed that is substantially free of C₉ aromatics for use in accordance with the disclosed methods typically will include numerous other hydrocarbons, many of which are only present in trace amounts. For example, the feed should be substantially free of non-aromatics such as, for example, paraffins and olefins. A feed that is substantially free of non-aromatics preferably comprises less than about 5 wt. % non-aromatics, and more preferably less than about 1 wt. % non-aromatics, based on the total weight of the feed. Although suitable feeds are preferably substantially free of non-aromatics, feeds containing non-aromatics can be processed by the disclosed methods as demonstrated in the examples reported below. The feed should be substantially free of sulfur (e.g., elemental sulfur and sulfur-containing hydrocarbons and non-hydrocarbons). A feed that is substantially free of sulfur preferably comprises less than about 1 wt. % sulfur, more preferably less than about 0.1 wt. % sulfur, and even more preferably less than about 0.01 wt. % sulfur, based on the total weight of the feed.

In various preferred embodiments, the feed is substantially free of xylene isomers, ethylbenzene, and/or benzene. A feed that is substantially free of xylene isomers preferably comprises less than about 3 wt. % xylene isomers, and more preferably less than about 1 wt. % xylene isomers, based on the total weight of the feed. Ahead that is substantially free ethylbenzene preferably comprises less than about 5 wt. % of ethylbenzene, and more preferably less than about 3 wt. % ethylbenzene, based on the total weight of the feed. A feed that is substantially free of ethylbenzene preferably comprises less than about 5 wt. % ethylbenzene, and more preferably less than about 3 wt. % ethylbenzene, based on the total weight of the feed.

In certain embodiments, after the feed is catalytically converted to a product containing xylene isomers, at least a portion of the xylene isomers is separated from the product. When separated out, the remaining product is lean in xylene isomers relative to the product just prior to the separation and, therefore, is referred to herein as a xylene-isomers lean product. Post separation, this xylene-isomers lean product can be recycled to the feed. Accordingly, in these embodiments, the method can be described as one in which the feed is catalytically converted to a product comprising xylene isomers, the xylene isomers are separated from the product, and the product is thereafter recycled to the feed. In these embodiments, the recycled product preferably contains small (or only trace) amounts of xylene isomers and contains predominantly unreacted feed, benzene, toluene, and C₉₊ aromatics.

In a further embodiment of the inventive method, the product contains xylene isomers and ethylbenzene present in a weight ratio of at least about 6 to 1, preferably at least about 10 to 1, and more preferably at least about 25 to 1. Stated another way, the method of converting a C₉ aromatics-comprising feed to a product containing xylene isomers includes contacting the feed with a suitable catalyst under conditions suitable to yield a weight ratio of xylene isomers to ethylbenzene in the product stream of at least about 6 to 1, preferably at least about 10 to 1, and more preferably at least about 25 to 1. Such a high weight ratio xylene isomers to ethylbenzene in the product stream is beneficial in downstream processing where the product is to be fractionated into its major constituents, i.e., into aromatics containing 6, 7, 8, and 9 carbons. Typically, further processing of a C₈ aromatics fraction would necessarily involve energy-consuming processing of the ethylbenzene to convert it to benzene (de-ethylation processes). These de-ethylation processes can cause yield losses of the xylene isomers. However, given the substantial absence of ethylbenzene in the liquid reaction product, and the accordingly substantial absence of ethylbenzene in the C₈ aromatics fraction, a much less energy-consuming processing can be used to rid the fraction of ethylbenzene. Additionally, the substantial absence of ethylbenzene means that downstream processes used to convert xylene isomers to para-xylene should not suffer yield losses of xylenes because de-ethylation processes are not necessary.

Moreover, the substantial absence of ethylbenzene is particularly desired. As previously noted, though ethylbenzene can be used as a raw material to make styrene, such ethylbenzene must be in a highly purified form. The particular ethylbenzene that results from disproportionating and transalkylating benzene, toluene, and C₉ aromatics is necessarily present in a mixture containing other aromatics. Separating ethylbenzene from such a mixture is very difficult and very expensive. Consequently, from a practical standpoint this ethylbenzene cannot be used in the manufacture of styrene. In practice, the ethylbenzene would either be used as a gasoline additive (as an octane booster therein) or likely be subjected to further disproportionation to yield light gas (e.g., ethane) and benzene. According to the invention, however, the substantial absence of ethylbenzene in the liquid reaction product and C₉ aromatics fraction would obviate such processing.

In another embodiment of the inventive method, the product contains xylene isomers to methylethylbenzene (MEB) in a weight ratio of at least about 1 to 1, preferably at least about 5 to 1, and more preferably at least about 10 to 1. Stated another way, the method of converting a C₉ aromatics-comprising feed to a product containing xylene isomers includes contacting the feed with a suitable catalyst under conditions suitable to yield a weight ratio of xylene isomers to methylethylbenzene in the product of at least about 1 to 1, preferably at least about 5 to 1, and more preferably at least about 10 to 1. The lack of (or low amounts of) methylethylbenzene in the product is advantageous in that there are lower amounts of such unreacted or produced C₉ aromatics that need to be recycled back to the feed for conversion, thus, conserving energy and reducing capital costs.

In yet another embodiment of the inventive method, the product contains xylene isomers to C₁₀ aromatics in a weight ratio of at least about 3 to 1, preferably at least about 5 to 1, and more preferably at least about 10 to 1. Stated another way, the method of converting a C₉ aromatics-comprising feed to a product containing xylene isomers includes contacting the feed with a suitable catalyst under conditions suitable to yield a weight ratio of xylene isomers to C₁₀ aromatics in the product of at least about 3 to 1, preferably at least about 5 to 1, and more preferably at least about 10 to 1. Such high ratios are evidence that the dominant reaction involving the C₉ aromatics is a disproportionation reaction yielding xylene isomers and not a reaction yielding C₁₀ aromatics, toluene, and benzene. The lack of or low amounts of C₁₀ aromatics in the product is advantageous in that there are lower amounts of such unreacted or produced C₁₀ aromatics that need to be recycled back to the feed for conversion, thus, conserving energy and reducing capital costs. To the extent that C₁₀ aromatics are present in the product, such C₁₀ aromatics are predominantly tetramethylbenzene, which can be recycled and are more amenable to conversion to xyiene isomers. Advantageously, the C₁₀ aromatics do not include much ethyldimethylbenzene and/or diethylbenzene, both of which are more difficult to convert to xylene isomers and, therefore, less likely to be recycled.

In a further embodiment of the inventive method, the product contains trimethylbenzene to methylethylbenzene in a weight ratio of at least about 1.5 to 1, preferably at least about 5 to 1, more preferably at least about 10 to 1, and even more preferably at least about 15 to 1. Stated another way, the method includes converting a C₉ aromatics-comprising feed to a product containing xylene isomers includes contacting the feed with a suitable catalyst under conditions suitable to yield a weight ratio of trimethylbenzene to methylethylbenzene in the product of at least about 1.5 to 1, preferably at least about 5 to 1, more preferably at least about 10 to 1, and even more preferably at least about 15 to 1. To obtain a xylene isomer from trimethylbenzene a single methyl group must be removed from the trimethylbenzene molecule. In contrast, to obtain a xylene isomer from methylethylbenzene, one must substitute a methyl group for the ethyl group on the benzene ring. Such a substitution is difficult to carry out. Consequently high ratios of trimethylbenzene to methylethylbenzene are advantageous in that trimethylbenzene is more amenable to conversion to xylene isomers than is methylethylbenzene and, consequently, is more amenable to recycle.

In a still further embodiment of the inventive method, the product contains benzene to ethylbenzene in a weight ratio of at least about 2 to 1, preferably at least about 5 to 1, and more preferably at least about 10 to 1. Stated another way, the method of converting a C₉ aromatics-comprising feed to a product containing xylene isomers includes contacting the feed with a catalyst under conditions suitable to yield a weight ratio of benzene to ethylbenzene in the product of at least about 2 to 1, preferably at least about 5 to 1, and more preferably at least about 10 to 1. Such high ratios are beneficial given that ethylbenzene of the type obtained during disproportionation and transalkylation reactions involving C₉ aromatics have lower value as a chemical feedstock given the difficulties in separating ethylbenzene from a mixture of other C₈ aromatics. As noted above, a molecule of a C₉ aromatic and benzene can be transalkylated to a molecule of xylene and toluene. Thus, the high ratio of benzene relative to ethylbenzene in the product can prove useful when considering that xylene lean portions of the product can be recycled to increase the yield of xylene isomers.

In another embodiment of the inventive method, the feed contains C₉ aromatics present in an amount (weight ratio) relative to the amount present in the product of at least about 1.5 to 1, preferably at least about 2 to 1, and more preferably at least about 4 to 1. Stated another way, the method of converting a C₉ aromatics-comprising feed to a product containing xylene isomers includes contacting the feed with a catalyst under conditions suitable to yield a weight ratio of C₉ aromatics present in the feed to that present in the product of at least about 1.5 to 1, preferably at least about 2 to 1, and more preferably at least about 4 to 1. Such a high conversion is beneficial in that there are lower amounts of unreacted C₉ aromatics that need to be recycled back to the feed for conversion, thus, conserving energy and reducing capital costs.

In yet another embodiment of the inventive method, the feed contains methylethylbenzene present in an amount (weight ratio) relative to the amount present in the product of at least about 2 to 1, preferably at least about 5 to 1, and more preferably at least about 10 to 1. Stated another way, the method of converting a C₉ aromatics-comprising feed to a product containing xylene isomers includes contacting the feed with a catalyst under conditions suitable to yield a weight ratio of methylethylbenzene present in the feed to that present in the product of at least about 2 to 1, preferably at least about 6 to 1, and more preferably at least about 10 to 1. Such a high ratio is evidence that the inventive method effectively converts a high proportion of the methylethylbenzene present among the C₉ aromatics in the feed. Indeed, the high ratios show that the reactions are effective to convert about 50%, preferably 90%, and most preferably 95% of the methylethylbenzene to light gas and lighter aromatics. Furthermore, such high ratios are evidence that the reactions do not yield methylethylbenzene.

The disclosed methods are generally illustrated in the sole drawing FIGURE, wherein an embodiment, generally designated 10, includes a reactor 12 and a liquid products separator 14, which typically is a distillation or fractionation tower/column. More specifically, a feed in a feed line 16 and a hydrogen-comprising gas in a gas line 18 are combined and heated in a furnace 20. The heated mixture is passed into the reactor 12 where the feed catalytically reacts in the presence of hydrogen to yield a product. The product exits the reactor 12 through a product line 22 and is thereafter cooled in a heat exchanger 24. A cooled, product exits the heat exchanger 24 via a transport line 26 and passes into a vessel 28 in which gas and liquids are separated from one another. As necessary (e.g., when transalkylating feeds containing C₉ aromatics), fresh hydrogen also can be passed directly into the reactor 12 via a gas line 18A. Gases, primarily hydrogen, are withdrawn from the vessel 28, and portions are compressed (compressor not shown), and recycled via a gas line 30 to the hydrogen-comprising gas in the gas line 18, while the remainder may be purged via a purge line 32. The liquids are withdrawn from the vessel 28 via a transport line 34 and passed into the liquids separator 14. Within the separator 14, constituents comprising the product are separated.

When the embodiment 10 is used for transalkylating feeds (in feed line 16) containing predominantly C₉ aromatics (and also containing some benzene and toluene), the major constituents comprising the product in the separator 14 will be xylene isomers and toluene. The xylene isomers exit the separator 14 via a product line 36. One or more recycle lines 38 and 40 may transport unconverted C₉ aromatics and the xylene isomers-lean product (typically containing toluene), respectively, back to the reactor 12, for example, by combining these products with fresh feed in the feed line 16. Lines 36A, 38A, and 40A may remain unused when transalkylating feeds containing predominantly C₉ aromatics; however, these lines can be used to recycle or purge certain constituents in the product as necessary.

When the embodiment 10 is used for disproportionating feeds (in feed line 16) containing predominantly toluene, the major constituents comprising the product in the separator 14 will be xylene isomers, toluene, and benzene. The xylene isomers exit the separator 14 via a product line 38A. A recycle line 36A can be used to transport toluene back to the reactor 12, for example, by combining this toluene with fresh feed in the feed line 16. Benzene may be removed from the process by a line 40A. Lines 36, 38, and 40 may remain unused when disproportionating feeds containing predominantly toluene; however, these lines can be used to recycle or purge certain constituents in the product as necessary.

Thus, entering the embodiment 10 are a feed (16) and a hydrogen-comprising gas (18), and exiting the process is a xylene isomers product (36 or 38A). Because the transalkylation and disproportionation performed in the process require a certain number of methyl groups to be present relative to the number of benzene groups, there may be some removal of the formed benzene and toluene out of the overall process.

Subsumed in the disclosed method (and the various embodiments thereof) is an understanding by those skilled in the art of suitable processing equipment and controls necessary to carry out the method. Such processing equipment includes, but is not limited to, appropriate piping, pumps, valves, unit operations equipment (e.g., reactor vessels with appropriate inlets and outlets, heat exchangers, separation units, etc.), associated process control equipment, and quality control equipment, if any. Any other processing equipment, especially where particularly preferred, is specified herein.

Generally, the disclosed method is carried out in a reaction vessel containing a non-sulfided catalyst suitable to convert the feed into a product containing xylene isomers. Suitable catalysts will generally include a support impregnated with a hydrogenation component, and the support will include a binder and a sieve, each of which are described in more detail below. Generally, the sieve contains sites active to convert the feed to xylene isomers. The catalyst should be designed such that the feed, recycle, and product can access these active sites and traverse the pores of the catalyst. The suitability of the catalyst will depend upon on a number of considerations. One such consideration is the size of the molecules contained in the feed, recycle, and product that can be expected to interact with the catalyst. Although active sites can be found on the exterior surfaces of the catalyst particle, most of the catalytically active sites will be present within the pores of the catalyst particle and, more specifically, within the pores of the sieve. Thus, molecules small enough to diffuse through (or traverse) the pores and reach the active sites can be catalytically converted therein to a product. If sufficiently small, the product also can traverse the pores and timely exit the reaction vessel. Molecules too large to traverse the pores, however, will pass around the catalyst and through the reactor unconverted because they will not fit into the pores where most of the catalytic sites are located. While there will be active sites on the outside surfaces of the catalyst (and not just within the pores), a porous material that does not permit diffusion of these large molecules is not ideally suited to perform the desired conversion. Similarly, product molecules formed within the pores may be so large that their transport out of the pores may be very slow, and they may convert to smaller (yet undesirable) molecules that diffuse more rapidly through the catalyst. Thus, the catalyst must contain pores of a sufficient size to accommodate not only the molecules within the feed, but also those that can be expected by the conversion.

As noted above, the disclosed methods contemplate feeds, recycles, and products containing a variety of molecules. The size of the molecules present in each will minimally determine the pore size of the sieve suitable for the catalyst. C₉₊ aromatics are, of course, larger than xylene isomers, toluene, and benzene. A sieve containing large pores at least about six angstroms to about eight angstroms) will permit C₉₊ aromatics to pass. In contrast, a sieve containing small pores (i.e., between about three angstroms and less than about four angstroms) generally will not permit any of these molecules to pass. Medium pore sieves will permit some of these molecules to pass, but not others. For example, C₉₊ aromatics generally will not pass through a sieve containing medium size pores (i.e., between about four angstroms and less than about six angstroms), while xylene isomers, toluene, benzene, and smaller molecules will pass through these sieves.

Sieves (also referred to herein as “molecular sieves”) suitable for use in the disclosed method include a wide variety of natural and synthetic, crystalline, porous oxides having channels, cages, and cavities of molecular dimensions. These sieves are typically formed from silica, alumina, and/or phosphorus oxide. Sieves preferred for use in accordance with the disclosed methods are those selected from the group consisting of aluminosilicates (also known as zeolites), aluminophosphates, silicoaluminophosphates, and mixtures thereof. Such sieves can have large pores or medium pores depending on the reactants, intermediates, and products likely to be encountered in the disclosed methods. For example, large pore sieves should be used when the feed, intermediates, or the product can be expected to include C₉₊ aromatics. Large pore sieves also can be used when the feed, intermediates, or the product can be expected to include molecules smaller in size than C₉₊ aromatics; however, a medium pore sieve may be equally or better suited than the large pore sieve in such instances. Suitable large pore sieves have a pore size of at least about six angstroms. Suitable medium pore sieves have a pore size of about four angstroms and less than about six angstroms. Generally, the support comprises about 20 wt. % to about 85 wt. % sieve, based on the total weight of the catalyst. As described in more detail below, however, the amount of sieve will be related to the amount of binder present in the support.

Examples of large pore zeolites include, but are not limited to, beta (BEA), EMT, FAU (e.g., zeolite X, zeolite Y (USY)), MAZ, mazzite, mordenite (MOR), zeolite L, LTL (IUPAC Commission of Zeolite Nomenclature). Preferred large-pore zeolites include beta (BEA), Y (USY), and mordenite (MOR) zeolites, general descriptions of each of which can be found in Kirk Othmer's “Encyclopedia of Chemical Technology,” 4^{th} Ed., Vol. 16, pp. 888-925 (John Wiley & Sons, New York, 1995) (hereafter “Kirk Othmer's Encyclopedia”), and W. M. Meier et al., “Atlas of Zeolite Structure Types,” A1-A5 and 1-16 (4^{th} Ed., Elsevier, 1996) (hereafter “Meier's Atlas”), the disclosures of which are incorporated by reference herein. Mixtures of these zeolites also are suitable. These zeolites can be obtained from commercial sources such as, for example, the Engelhard Corporation (Iselin, N.J.), PQ Corporation (Valley Forge, Pa.), Tosoh USA, Inc. (Grove City, Ohio), and UOP Inc. (Des Plaines, Ill.). More preferably, the large-pore zeolite for use in the invention is a mordenite zeolite. Examples of large pore aluminophosphates include, but are not limited to SAPO-37 and VFI. Mixtures of these aluminophosphates also are suitable.

Examples of medium pore zeolites include, but are not limited to, Edinburgh University-one (EUO), ferrierite (FER), Mobil-Eleven (MEL), Mobil-fifty seven (MFS), Mobil-Five (MFI), Mobil-twenty three (MTT), new-eighty seven (NES), theta-one (TON), and mixtures thereof. Preferably, however, medium pore zeolites include Mobil-Five (MFI) and Mobil-Eleven (MEL). Preferable Mobil-Five (MFI) zeolites include those selected from the group consisting of ZSM-5, silicalite, related isotypic structures thereof, and mixtures thereof. Preferable Mobil-Eleven (MEL) zeolites include those selected from the group consisting of ZSM-11, related isotypic structures, and mixtures thereof. General descriptions of medium pore zeolites can be found in Kirk Othmer's Encyclopedia and in Meier's Atlas, the disclosures of which are incorporated by reference herein. These types of zeolites can be obtained from commercial sources such as, for example, ExxonMobil Chemical Company (Baytown, Tex.), Zeolyst International (Valley Forge, Pa.), and UOP Inc, (Des Plaines, Ill.). An example of a suitable medium pore aluminophosphate is aluminophosphate-elevent (AEL).

As noted above, the support includes a sieve and a macroporous binder. The “micro,” “meso,” and “macro” prefaces to the terms “pore,” “pore volume,” and “porous” are well known to those having ordinary skill in the art of making and using catalysts. In this art, micropore generally refers to the volume of pores having radii measuring about twenty angstroms (two nanometers (nm)) or less. Mesopore generally refers to the volume of pores having radii measuring greater than about twenty angstroms (two nm) and less than about 500 angstroms (50 nm). Macropore generally refers to the volume of the pores having radii measuring greater than about 500 angstroms (50 nm). See e.g., S. M. Auerbach, “Handbook of Zeolite Science and Technology,” 291 (Marcel Dekker, Inc., New York, 2003).

Suitable macroporous binders include, but are not limited to, aluminas, aluminum phosphates, clays, silica-aluminas, silicas, silicates, titanias, zirconias, and mixtures thereof. Certain of these binders also can offer advantages in terms of easy attainment of suitable physical properties by steaming to increase average pore diameter without appreciably decreasing pore volume, as described in more detail below. Preferred aluminas include γ-alumina, η-alumina, pseudobohemite, and mixtures thereof. Generally, the support can include up to about 50 wt. % binder, based on the total weight of the support, and preferably includes about 10 wt. % to about 30 wt. % binder, based on the total weight of the support. The weight ratio of the sieve to the binder preferably is about 20:1 to about 1:10, and more preferably about 10:1 to about 1:2.

The catalyst preferably is bifunctional in that it includes as active sites not only those of an acid in the sieve, but also those of a hydrogenation component. Accordingly, the catalyst also includes a hydrogenation component. When incorporated into the catalyst, the hydrogenation component assists in converting the feed into a product containing xylene isomers. More specifically, the hydrogenation component catalyzes a reaction between molecular hydrogen and free olefins that may be present in the reactor to prevent the olefins from deactivating the catalytic (acid) sites in the sieve. The molecular hydrogen is believed to saturate the olefins so that the olefins cannot react with aromatics at the catalytic (acid) sites to form undesired heavy by products.

Preferably, the hydrogenation component is a metal or metal oxide. The metal preferably is selected from the group consisting of Group VIB metals, Group VIIB metals, Group VIII metals, and combinations thereof. Among this group, metals from Group VIB are preferred. Preferably Group VIB metals include, but are not limited to, chromium, molybdenum, tungsten, and combinations thereof. The Group VIB metal oxide preferably is selected from the group consisting of molybdenum oxides, chromium oxides, tungsten oxides, and combinations of any two or more thereof wherein the oxidation state of the metal can be any available oxidation state. For example, in the case of a molybdenum oxide, the oxidation state of molybdenum can be 0, 2, 3, 4, 5, 6, or combinations of any two or more thereof.

Examples of suitable Group VIB metal compounds include, but are not limited to, chromium-, molybdenum-, and/or tungsten-containing compounds. Suitable chromium-containing compounds include, but are not limited to, chromium(II) acetate, chromium(II) chloride, chromium(II) fluoride, chromium(III) 2,4-pentanedionate, chromium(III) acetate, chromium(III) acetylacetonate, chromium(III) chloride, chromium(III) fluoride, chromium hexacarbonyl, chromium(III) nitrate, and chromium(III) perchlorate. Suitable tungsten-containing compounds include, but are not limited to, tungstic acid, tungsten(V) bromide, tungsten(IV) chloride, tungsten(VI) chloride, tungsten hexacarbonyl, and tungsten(VI) oxychloride. Molybdenum-containing compounds are the preferred metal and such compounds include, but are not limited to, ammonium dimolybdate, ammonium heptamolybdate(VI), ammonium molybdate, ammonium phosphomolybdate, bis(acetylacetonate)dioxomolybdenum(VI), molybdenum fluoride, molybdenum hexacarbonyl, molybdenum oxychloride, molybdenum(II) acetate, molybdenum(II) chloride, molybdenum(III) bromide, molybdenum(III) chloride, molybdenum(IV) chloride, molybdenum(V) chloride, molybdenum(VI) fluoride, molybdenum(VI) oxychloride, molybdenum(VI) tetrachloride oxide, potassium molybdate, and molybdenum oxides in which the oxidation state of molybdenum can be 2, 3, 4, 5, and 6, and combinations of two or more thereof. Preferably, the Group VIB metal compound is an ammonium molybdate due to its abundance and the relative ease with which molybdenum can be incorporated into the preferred sieves.

Examples of suitable Group VIIB metal compounds include, but are not limited to, rhenium-containing metal compounds, such as, for example, (NH₄)ReO₄, Re₂O₇, ReO₂, ReCl₃, ReCl₅, Re(CO)₅Cl, Re(CO)₅Br, Re₂(CO)₁₀, and combinations thereof. Examples of suitable Group VII metal compounds include, but are not limited to, nickel-, palladium- and platinum-containing compounds. Examples of nickel-containing metal compounds include, but are not limited to, nickel chloride, nickel bromide, nickel nitrate, and nickel hydroxide. Examples of palladium-containing metal compounds include, but are not limited to, palladium chloride, palladium nitrate, palladium acetate, and palladium hydroxide. Examples of platinum-containing metal compounds include, but are not limited to, chloroplatinic acid (H₂PtCl₆.xH₂O), hexachloroplatinic(IV) acid, platinum (II) or (IV) chloride (platinic chloride), platinum (ID or (IV) bromide, (II) iodide, cis- or trans-diamine platinum(II) chloride, cis- or trans-diamine platinum (IV) chloride, diammine platinum(II) nitrite, (ethylenediamine) platinum(II) chloride, tetramine platinum (II) chloride or chloride hydrate (Pt(NH₃)₄Cl₂.H₂O or Pt(NH₃)₄Cl₂), tetramine platinum(II) nitrate, (ethylenediamine) platinum (II) chloride, tetramine platinum (II) nitrate (Pt(NH₃)₄(NO₃)₂), tetrakis(triphenylphosphine) platinum (0), cis- or trans-bis(triethylphosphine) platinum (II) chloride, cis- or trans-bis(triethylphosphine) platinum (II) oxalate, cis-bis(triphenylphosphine) platinum (II) chloride, bis(triphenylphosphine) platinum(IV)oxide, (2,2′-6′,2″-terpyridine) platinum (IT) chloride dihydrate, cis-bis(acetonitrile) platinum dichloride, cis-bis(benzonitrile) platinum dichloride, platinum(II) acetylacetonate, (1c,5c-cyclooctadiene) platinum (II) chloride or bromide, platinum nitrosyl nitrate, and tetrachlorodiamine platinum (IV). Other Group VIII metals compounds that can be used include cobalt-, rhodium-, iridium-, and ruthenium-containing compounds.

The amount of the hydrogenation component (e.g., metal or metal oxide) present in the catalyst should be sufficient to be effective with transalkylation, dealkylation, and disproportionation processes. Accordingly, the amount of the hydrogenation component preferably is in a range of about 0.1 wt. % to about 20 wt. %, more preferably about 0.5 wt. % to about 10 wt. %, and even more preferably about 1 wt. % to 5 wt. %, based on the total weight of the catalyst. Molybdenum is the preferred metal and, preferably the support is impregregnated with ammonium heptamolybdate. Accordingly, the catalyst preferably includes about 0.5 wt. % to about 10 wt. % molybdenum or molybdenum oxide, more preferably about 1 wt. % to about 5 wt. % molybdenum or molybdenum oxide, and even more preferably about 2 wt. % molybdenum or molybdenum oxide, based on the total weight of the catalyst. If a combination of metals or metal oxides is used, the molar ratio of the second, third, and fourth metal oxides to the first metal oxide should be in a range of about 1:100 to about 100:1.

Any methods suitable for incorporating a metal or metal oxide into catalyst support such as, for example, impregnation or adsorption, can be used to make the catalyst. For example, the support can be prepared by mixing the sieve and binder by stirring, blending, kneading, or extrusion. Preferably, the mixing occurs under atmospheric pressure, but can occur at pressures slightly above and below atmospheric pressure. The obtained mixture then can be dried in air at a temperature in the range of from about 20° C. to about 200° C., preferably about 25° C. to about 175° C., and more preferably 25° C. to 150° C. for about 0.5 hour to about 50 hours, preferably about one hour to about 30 hours, and more preferably one hour to 20 hours. After the sieve and binder are sufficiently mixed and dried (to form an extrudate, for example), the support optionally can be calcined in air at a temperature in a range of about 200° C. to 1000° C., preferably about 250° C. to about 750° C., and more preferably about 350° C. to about 650° C. The calcination can be carried out for about 1 hour to about 30 hours, and more preferably about 2 hours to about 15 hours, to yield a calcined support.

The metal or metal oxide can be incorporated into the prepared support or, it can be incorporated into the sieve/binder mixture used to form the support. Where the binder is combined with a metal compound, it can be subsequently converted to a metal oxide by heating at elevated temperature, generally in air. As noted above, the metal or metal oxide preferably is selected from the Group VIB metals, such as, chromium, molybdenum, tungsten, and combinations and oxides thereof. The metal compound can be dissolved in a solvent before being contacted with the support. Preferably, however, the metal compound is an aqueous solution. The contacting can be carried out at any temperature and pressure. Preferably, however, the contacting occurs at a temperature in a range of about 15° C. to about 100° C., more preferably about 20° C. to about 100° C., and even more preferably about 20° C. to about 60° C. The contacting preferably also occurs at atmospheric pressure, for a length of time sufficient to ensure that the metal oxide has been incorporated into the support. Generally, this length of time is about 1 minute to about 15 hours, and preferably about 1 minute to about 5 hours.

As described in more detail below, the catalyst (and support) can be prepared to include macropores by, for example, utilizing a pore former when preparing the catalyst (and support), utilizing a binder that contains such macropores (i.e., a macroporous binder), or exposing the catalyst to heat (in the presence or absence of steam). A pore former is a material capable of assisting in the formation of pores in the catalyst support such that the support contains more and/or larger pores than if no pore former was used in preparing the support. The methods and materials necessary to ensure suitable pore size are generally known by persons having ordinary skill in the art of preparing catalysts. Examples of pore formers are disclosed in Doyle et al. U.S. patent application publication No. 2004/00220047 A1, the disclosure of which is incorporated herein by reference. Examples of suitable pore formers include, but are not limited to, acids, anionic surfactants, cationic surfactants, polysaccharides, waxes, and mixtures thereof. Suitable acids include citric acid, lactic acid, oxalic acid, stearic acid, tartaric acid, and mixtures thereof. Suitable anionic surfactants include sodium alkylbenzenesulfonates, alkyl ethoxy sulfates, alkyl sulfates, ammonium carbonate ((NH₄)₂CO₃), silicone carboxylates, silicone phosphate esters, silicone sulfates, and mixtures thereof. Suitable cationic surfactants include silicone amides, silicone amido quaternary amines, silicone imidazoline quaternary amines, tallow trimethylammonium chloride, and mixtures thereof. Polysaccharides suitable for use as pore formers include carboxylmethyl cellulose, cellulose, cellulose acetate, methylcellulose, polyethylene glycol, starch, walnut powder, and mixtures thereof. Waxes suitable for use as pore formers include microcrystalline wax, montan wax, paraffin wax, polyethylene wax, and mixtures thereof. Preferably, the pore former is mixed with the binder to provide a more uniform distribution of the pore former within the binder and, therefore, to ensure a macroporous binder.

The catalyst can be heated to attain pores having an average radius of greater than about 500 angstroms (50 nm). This heating step can be performed in the absence of steam or in the presence of steam, which is also known as steaming, steam treating, or steam treatment. Steaming can be performed as an alternative to the use of the pore former or as an additional step when a pore former was already used. Generally, steam treating a catalyst can desirably increase the average pore diameter without appreciably decreasing pore volume to result in a catalyst suitable for use in accordance with the disclosed methods. Such steam treatments are generally known by persons having ordinary skill in the art of making catalysts and are generally disclosed in, for example, U.S. Pat. No. 4,395,328, the disclosure of which is incorporated herein by reference. Preferably, the catalyst is heated in the presence of steam at sufficient elevated temperature, steam pressure, and time period to increase the average pore diameter of a shaped catalyst in the absence of any appreciable reduction in pore volume. Preferably, the steam is employed at a pressure of about 30 kPa (4.4 psig) to about 274 kPa (25 psig). The time during which the catalyst is contacted with steam is about fifteen minutes to about three hours, and preferably about 30 minutes to about two hours. The elevated temperature at which the steam treatment is performed is about 704° C. (1,300° F.) to about 927° C. (1,700° F.), preferably about 760° C. (1,400° F.) to about 871° C. (1,600° F.). While specific combinations of values for these steaming conditions are not provided, any combination that will provide an increase in the average pore diameter without any appreciable change in pore volume is suitable. Steam treatment also can be utilized to improve the selectivity, attrition resistance, and heat stability of a catalyst.

Pore volume distributions and macropore volume of catalysts can be determined by techniques known by those having ordinary skill in the art of catalyst preparation. Mercury intrusion porosimetry (e.g., ASTM D4284-03), which is a widely-known technique to measure the pore-size distribution of porous materials, is one suitable technique. According to this technique, small samples (e.g., about 0.25 grams to about 0.5 grams) are first evacuated, and then surrounded by a mercury bath. Mercury is a non-wetting fluid for most porous materials of interest, so increasing pressure is required to force the mercury into smaller and smaller pores. In the porosimeter (e.g., a Quantachrome Poremaster 60 porosimeter manufactured by Quantachrome Instruments of Boynton Beach, Fla.), the volume of mercury that goes into the sample is monitored as the pressure is increased. This volume of mercury is then associated with a pore diameter that is determined by assuming a circular, cylindrical pore geometry. As the pressure is increased, a wide range of pore sizes can be explored. Porosimetry measurements can be calculated with the Washburn equation:

PD=−4γ cos(θ).

where P is the applied pressure, D is the diameter, γ is the surface tension of mercury (480 dynes per centimeter) and θ is the contact angle between mercury and the pore wall (average value being 140°). Preferably, the macropore volume of the catalyst is about 0.02 cubic centimeters per gram (cc/g) to about 0.5 cc/g, more preferably about 0.05 cc/g to about 0.35 cc/g, and even more preferably about 0.1 cc/g to about 0.3 cc/g.

While both fixed- and expanded-bed processes are contemplated herein, fixed bed processes are preferred. In fixed-bed processes, the feed and a hydrogen-containing gas are passed downwardly through a packed bed of catalyst under conditions temperature, pressure, hydrogen flow rate, space velocity, etc., that vary somewhat depending on the choice of feed, reactor capacity, and other factors known to those having ordinary skill in the art. Where the reactions can be expected to require or produce heat, fixed bed reactors can comprise multiple tubes each packed with a catalyst through which the reactants and products will pass. On the shell side of the tubes, a heat transfer medium can be used to provide or dissipate heat. Toluene disproportionation reactions generally are isothermal, while transalkylation reactions generally are slightly exothermic. Thus, fixed-bed reactors utilizing such tubes and heat transfer media can be useful to better control and/or optimize the temperature of the reactions.

The crush-strength (or durability) of the catalyst is important in fixed bed operations because of the pressure drop resulting from passage of the feed and hydrogen-containing gas through the packed catalyst bed. The size and shape of the catalyst also can be important in fixed bed operations because of their effect not only on pressure drop through the bed but also on catalyst-loading and contact between the catalyst and feed components. Use of larger catalyst particles near the top of a catalyst bed and smaller particles throughout the remainder of the bed can advantageously lead to decreased pressure drop. Catalyst in the form of spheres or extrudate, preferably about 0.01 inch (0.25 mm) to about 0.1 inch (2.5 mm) in diameter, should promote suitable contact between catalyst and feed components while avoiding excessive pressure drop through a catalyst bed. More preferably, the catalyst particles have an average particle size of about 1/32-inch (0.8 mm) to about 1/12-inch (2.1 mm) diameter. Trilobe, cloverleaf, cross, and “C”-shaped catalysts common in the art should perform suitably in terms of maximizing catalyst efficiency and promoting a high level of contact between catalyst and the feed constituents.

Other physical properties that are not critical with respect to catalyst activity but may influence performance include bulk density, mechanical strength, abrasion resistance, and average particle size. The bulk density of the catalyst preferably is about 0.3 g/cc to about 0.5 g/cc. Mechanical strength should be at least high enough to permit use in a given process without undesirable fragmentation or other damage. Similarly, abrasion resistance should be high enough to permit the catalyst particles to withstand particle to particle contact as well as contact between particles and reaction zone internals, particularly in expanded bed processes. Preferably, crush strength of the catalyst composition is such that a particle ⅛-inch (3.2 mm) in length and 1/32-inch (0.8 mm) in diameter will withstand at least about three pounds of pressure. Particle size can vary somewhat depending on the particular process to be used. The shape of the particles, however, can vary widely, as noted above, depending on process requirements.

Depending on the severity of operation and other process parameters, the catalyst will age. As the catalyst ages, its activity for the desired reactions tends to slowly diminish due to the formation of coke deposition or feed poisons on the surfaces of the catalyst. The catalyst may be maintained at or periodically regenerated to its initial level of activity by methods generally known by persons having ordinary skill in the art. Alternatively, the aged catalyst may simply be replaced with new catalyst.

To the extent that the aged catalyst is not replaced with new catalyst, the aged catalyst may require regeneration as frequently as once every two years, as often as once every year, or, on occasion, as often as once every six months. As used herein, the term “regeneration” means the recovery of at least a portion of the molecular sieve initial activity by combusting any coke deposits on the catalyst with oxygen or an oxygen-containing gas. The literature is replete with catalyst regeneration methods that can be used in the process of the present invention. Some of these regeneration methods involve chemical methods for increasing the activity of deactivated molecular sieves. Other regeneration methods relate to methods of regenerating coke-deactivated catalysts by the combustion of the coke with an oxygen-containing gas stream such as, for example, a cyclic flow of regeneration gases or the continuous circulation of an inert gas containing a quantity of oxygen in a closed loop arrangement through the catalyst bed.

The catalyst for use in the disclosed method is particularly suited for regeneration by the oxidation or burning of catalyst deactivating carbonaceous deposits (also known as coke) with oxygen or an oxygen-containing gas. Although the methods by which a catalyst may be regenerated by coke combustion can vary, preferably it is performed at conditions of temperature, pressure, and gas space velocity, for example, which are least thermally damaging to the catalyst being regenerated. It is also preferable to perform the regeneration in a timely manner to reduce process down-time in the case of a fixed bed reactor system or equipment size, in the case of a continuous regeneration process. A spare reactor can be provided to minimize the process down-time in a multi-reactor configuration. For example, in a multi-reactor configuration utilizing five reactors, at any given time, one of the reactors may be in a regeneration mode and off-line relative to the process. That off-line reactor can be brought on-line while another is brought off-line with minimum disturbance/interruption to the overall process.

Although the optimum regeneration conditions and methods are generally known by persons having ordinary skill in the art, catalyst regeneration preferably is accomplished at conditions including a temperature range of about 550° F. (about 287° C.) to about 1300° F. (about 705° C.), a pressure range of about zero pounds per square inch gauge (psig) (about zero mega-Pascals (MPa)) to about 300 psig (about two MPa), and a regeneration gas oxygen content of from about 0.1 mole percent to about 25 mole percent. The oxygen content of the regeneration gas typically can be increased during the course of a catalyst regeneration procedure based on catalyst bed outlet temperatures to regenerate the catalyst as quickly as possible while avoiding catalyst-damaging process conditions. The preferred catalyst regeneration conditions include a temperature ranging from about 600° F. (about 315° C.) to about 1150° F. (about 620° C.), a pressure ranging from about zero psig (about zero MPa) to about 150 psig (about one MPa), and a regeneration gas oxygen content of about 0.1 mole percent to about 10 mole percent. The oxygen-containing regeneration gas generally includes nitrogen and carbon combustion products such as carbon monoxide and carbon dioxide, to which oxygen in the form of air has been added. However, it is possible that the oxygen can be introduced into the regeneration gas as pure oxygen, or as a mixture of oxygen diluted with another gaseous component. Preferably the oxygen-containing gas is air.

Conditions suitable for carrying out the process of the invention can include a weight hourly space velocity (WHSV) of the fluid feed stream in the range of about 0.1 to about 30, preferably about 0.5 to about 20, and most preferably about 1 to about 10 unit mass of feed per unit mass of catalyst per hour. The hydrogen-comprising gas (e.g., molecular hydrogen) is present in a molar ratio relative to hydrocarbons in the feed of about 0.1:1 to about 10:1, preferably about 0.5:1 to about 8:1, and more preferably about 1:1 to about 6:1.

Generally, the pressure can be in a range of about 0.17 MPa (about 25 psi) to about 6.9 MPa (about 1000 psi), preferably about 0.34 MPa (about 50 psi) to about 4.1 MPa (about 600 psi), and more preferably about 0.69 MPa (about 100 psi) to about 2.76 MPa (about 400 psi). The temperature suitable for carrying out the process of the invention is in a range of about 200° C. (about 392° F.) to about 800° C. (about 1472° F.), more preferably about 300° C. (about 572° F.) to about 600° C. (about 1112° F.), and even more preferably about 350° C. (about 662° F.) to about 500° C. (about 932° F.).

Sufficient porosity is believed to be important from the standpoint of attaining high exposure of reactants to catalytically active sites, while an appreciable macropore volume is believed to be necessary to ensure access to the sites and activity maintenance. If pore volume is too high, however, the mechanical strength and bulk density of the catalyst can suffer. Thus, a suitable balance should be attained to ensure that the catalyst best serves its intended purpose.

### EXAMPLES

The following examples are presented to facilitate a better understanding of the disclosed methods. They are presented for the purpose of illustration only and are not intended to limit the scope of the claimed methods. In all examples, micropore volume and pore size distribution (when determined) were determined by nitrogen desorption, and macropore volume was determined by mercury penetration using a mercury porosimeter. Example 1 illustrates the conversion of a nitration-grade toluene feed to a product containing benzene and xylene isomers with a catalyst support lacking macropores. For the sake of comparison, Examples 2 through 8 illustrate the conversion of a similar feed to a product containing benzene and xylene isomers with a catalyst support containing macropores. The comparison shows that the catalyst support containing macropores results in improved conversion determinable by higher toluene conversion and higher selectivity for xylene isomers. Example 9 illustrates the conversion of nitration-grade toluene with a catalyst lacking macropore volume and one possessing macropore volume, and the stability of each catalyst.

Examples 10 and 11 illustrate the ability of a molybdenum oxide-impregnated macroporous catalysts to convert a feed containing toluene, benzene, and some light non-aromatics. Examples 12 and 13 illustrate the ability of molybdenum oxide-impregnated macroporous catalysts to convert feeds containing C₉₊ aromatics. Examples 14 and 15 illustrate the ability of molybdenum oxide-impregnated macroporous catalysts to convert feeds predominantly containing C₉ aromatics. Example 16 illustrates the ability of a molybdenum oxide-impregnated macroporous catalyst to convert a feed containing toluene and C₉ aromatics. Example 17 illustrates the ability of a molybdenum oxide-impregnated macroporous catalyst to convert a feed containing toluene and C₉₊ aromatics. Collectively, these examples demonstrate that the disclosed methods can accommodate various feeds and can recycle almost any byproduct without significant process modifications and without the necessity of replacing the catalyst.

### Catalyst Preparation

A number of different catalysts were prepared and tested as described below. For comparative purposes, catalysts “X” and “H” contained supports having insignificant amounts of macropore volume, whereas the other catalysts contained supports having significant amounts of macropore volume.

Catalyst “X” was prepared with H-mordenite zeolite (commercially-available from Engelhard Corporation (Iselin, N.J.)) having an Si/Al ratio of 41.6 and a sodium (Na) level of 130 parts per million (ppm). The support was prepared by mixing the zeolite with an alumina binder to form a slurry. The slurry was then extruded to form 1/12-inch cylindrical pellets (80% sieve/20% binder) and then calcined. An aqueous solution of ammonium heptamolybdate was then mixed with and impregnated on the extrudate to give a mordenite catalyst having 2% molybdenum distributed evenly throughout. The impregnated catalyst was then calcined at about 500° C. for about one hour to about three hours. The macropore volume greater than about 50 nm for catalyst “X” was determined by mercury adsorption techniques to be 0.018 cc/g.

Catalysts “A” through “G” were prepared with H-mordenite zeolite (commercially-available from Engelhard Corporation) having an Si/Al ratio of 41.6 and a sodium (Na) level of 130 parts per million (ppm). The support was prepared by mixing the zeolite with an alumina binder to form a slurry. A pore forming reagent was added to the slurry, and the resulting mixture was then extruded to form either 1/12-inch cylindrical pellets or 1/16-inch trilobe pellets (80% sieve/20% binder). (With respect to Catalyst “E,” the resulting mixture was then extruded to form 1/12-inch cylindrical pellets (70% sieve/30% binder)). Table 1, below, indicates the extruded pellets for each catalyst. The extrudate was then calcined, and the pore forming reagent was decomposed by the thermal treatment. An aqueous solution of ammonium heptamolybdate was then mixed with and impregnated on the extrudate to give a mordenite catalyst having 2% molybdenum distributed evenly throughout. The impregnated catalyst was then calcined at about 500° C. for about one hour to about three hours. The macropore volume greater than about 50 nm for these catalysts was determined by mercury adsorption techniques and is reported in Table 1, below, for each catalyst.

Catalyst “H” was prepared with a lab-synthesized Na-mordenite zeolite (commercially-available from Engelhard Corporation). The zeolite was ion exchanged to provide an H-mordenite zeolite having an Si/Al ratio of 36.1 and a sodium (Na) level of 260 parts per million (ppm). The support was prepared by mixing the zeolite with an alumina binder to form a slurry. A pore forming reagent was added to the slurry, and the resulting mixture was then extruded to form 1/16-inch trilobe pellets (80% sieve/20% binder). An aqueous solution of ammonium heptamolybdate was then mixed with and impregnated on the extrudate to give a mordenite catalyst having 2% molybdenum distributed evenly throughout. The macropore volume greater than about 50 nm for catalyst “H” was determined by mercury adsorption techniques to be 0.01 cc/g.

Catalyst “I” was prepared with a lab-synthesized Na-mordenite zeolite (commercially-available from Engelhard Corporation). The zeolite was ion exchanged to provide an H-mordenite zeolite having an Si/Al ratio of 36.1 and a sodium (Na) level of 260 parts per million (ppm). The support was prepared by mixing the zeolite with an alumina binder to form a slurry. The slurry was then extruded to form 1/16-inch trilobe pellets (80% sieve/20% binder). The extrudate was then calcined, and the pore forming reagent was decomposed by the thermal treatment. An aqueous solution of ammonium heptamolybdate was then mixed with and impregnated on the extrudate to give a mordenite catalyst having 2% molybdenum distributed evenly throughout. The macropore volume greater than about 50 nm for catalyst “I” was determined by mercury adsorption techniques to be 0.18 cc/g.

TABLE 1

| Catalyst | Size & Shape | Hg Macropore Vol. (>50 nm) (cc/g) |
|---|---|---|
| X | 1/12″ cylinder | 0.018 |
| A | 1/16″ Trilobe | 0.132 |
| B | 1/12″ cylinder | 0.255 |
| C | 1/12″ cylinder | 0.288 |
| D | 1/12″ cylinder | 0.280 |
| E | 1/12″ cylinder | 0.212 |
| F | 1/12″ cylinder | 0.281 |
| G | 1/16″ Trilobe | 0.280 |
| H | 1/16″ Trilobe | 0.01 |
| I | 1/16″ Trilobe | 0.18 |

### Pilot Plant Experimental Runs

The performance of each catalyst was separately evaluated in an automated continuous-flow, fixed-bed pilot plant. In each run, ten grams of the subject catalyst were packed in the reactor, which generally was a pipe having an inlet and outlet. The catalysts were pre-treated with flowing hydrogen for two hours at 750° F. (400° C.) and 200 psig (1.38 MPa) prior to the introduction of a (liquid) feed. The feed consisted of a mixture of hydrogen to hydrocarbon gas in a 4:1 molar ratio. Unless stated otherwise herein, the reactor conditions were set at a temperature of about 750° F. (400° C.) and a pressure of about 200 psig (1.38 MPa). The weight hourly space velocity (WHSV) was either 4.0 or 6.0 (corresponding to liquid feed flow rates of about 40 grams per hour (g/hr) and 60 g/hr, respectively) as indicated herein. Unless stated otherwise herein, the catalysts were used at constant conditions for a minimum of three to five days prior to obtaining product samples to ensure stable performance. Assuming first order kinetics for an equilibrium reaction, the activity of catalyst “X” (described above) was taken as 1.0.

### Example 1

This example illustrates the performance capabilities of a catalyst lacking macropore volume (catalyst “X”) to convert nitration-grade toluene to a product comprising xylene isomers. Separate runs were conducted with identical feeds at a WHSV of 4.0 and at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and products (Pdt. Wt. %) obtained in each run are shown in Table 2, below.

TABLE 2
Catalyst “X”

| | Feed Wt % | Pdt. Wt % | |
|---|---|---|---|
| WHSV | | 4.0 | 6.0 |
| Non-aromatics | 0.08 | 2.28 | 1.50 |
| Benzene | 0.00 | 15.73 | 12.81 |
| Toluene | 99.83 | 60.91 | 68.29 |
| Ethylbenzene | 0.05 | 0.18 | 0.13 |
| p-Xylene | 0.02 | 4.38 | 3.71 |
| m-Xylene | 0.03 | 9.49 | 8.00 |
| o-Xylene | 0.00 | 4.16 | 3.49 |
| Propylbenzene | 0.00 | 0.00 | 0.00 |
| Methylethylbenzene | 0.00 | 0.27 | 0.22 |
| Trimethylbenzene | 0.00 | 2.33 | 1.66 |
| A₁₀₊ | 0.00 | 0.26 | 0.18 |

The conversion of toluene is determined by dividing the difference in the amount of toluene in the feed and product by the toluene present in the feed. For example, using the data obtained from the run with catalyst “X” and a WHSV of 4.0, the toluene conversion was about 39.0 (i.e., 39.0=100×(99.83−60.91)+99.83). In contrast, using the data obtained from the run with catalyst “X” and a WHSV of 6.0, the toluene conversion was about 31.6 (i.e., 31.6=100×(99.83−68.29)+99.83).

The selectivity of any particular constituent in the product is determined by dividing the yield of the constitutent by the conversion of toluene. Thus, for example, using the data obtained from the run with catalyst “X” and a WHSV of 4.0, the benzene selectivity was about 40.3% (i.e., 40.3=100×(15.73 4+39.0)), and the xylene isomers selectivity was about 46.2% (i.e., 46.2=100×18.3+39.0)). In contrast, using the data obtained from the run with catalyst “X” and a WHSV of 6.0, the benzene selectivity was about 40.5% (i.e., 40.5=100×(12.81+31.6)), and the xylene isomers selectivity was about 48.1% (i.e., 48.1=100×15.2+31.6)). Additionally, the selectivity of C₉₊ aromatics at WSHV of 4.0 and 6.0 was 7.3% and 6.6%, respectively.

At WSHV of 4.0, there is 15.7% benzene and 18.0% xylene isomers (0.87 weight ratio) as the major products. The ethylbenzene present in the product at WHSV of 4.0 comprises about 0.99 wt. % of the C₈ aromatics, based on the total weight of the C₈ aromatics. At WSHV of 6.0, there is 12.8% benzene and 15.2% xylene isomers (0.84 weight ratio) as the major products. The ethylbenzene present in the product at WHSV of 6.0 comprises about 0.85 wt % of the C₈ aromatics, based on the total weight of the C₈ aromatics.

### Example 2

This example illustrates the performance capabilities of a catalyst containing macropores, catalyst “A,” to convert nitration-grade toluene to a product comprising xylene isomers. Separate runs were conducted with nearly-identical feeds at a WHSV of 4.0 and at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and products (Pdt. Wt. %) obtained in each run are shown in Table 3, below.

TABLE 3
Catalyst “A”

| | Feed | Product | Feed | Product |
|---|---|---|---|---|
| WHSV | 4.0 | | 6.0 | |
| Non-aromatics | 0.08 | 0.66 | 0.08 | 0.57 |
| Benzene | 0.00 | 18.32 | 0.00 | 15.76 |
| Toluene | 99.82 | 55.90 | 99.81 | 61.90 |
| Ethylbenzene | 0.05 | 0.22 | 0.05 | 0.17 |
| p-Xylene | 0.02 | 5.16 | 0.02 | 4.57 |
| m-Xylene | 0.03 | 11.25 | 0.03 | 9.93 |
| o-Xylene | 0.00 | 4.94 | 0.00 | 4.34 |
| Propylbenzene | 0.00 | 0.00 | 0.00 | 0.00 |
| Methylethylbenzene | 0.00 | 0.28 | 0.00 | 0.28 |
| Trimethylbenzene | 0.00 | 2.97 | 0.00 | 2.22 |
| A₁₀₊ | 0.00 | 0.30 | 0.01 | 0.25 |

At WSHV of 4.0, there is 18.3% benzene and 21.4% xylene isomers (0.86 weight ratio) as the major products. At WSHV of 6.0, there is 15.8% benzene and 18.8% xylene isomers (0.84 weight ratio) as the major products. Based on the obtained data shown in Table 3, the toluene conversions at WSHV of 4.0 and 6.0 was 44.0%, and 38.0% respectively. In contrast, the toluene conversions of a similar feed at WSHV of 4.0 and 6.0 utilizing catalyst “X” was 39.0%, and 31.6%, respectively. Similarly, the xylene isomers selectivity with catalyst “A” at WSHV of 4.0 and 6.0 was 48.5%, and 49.6%, respectively. In contrast, the xylene isomers selectivity with catalyst “X” at WSHV of 4.0 and 6.0 was 46.2%, and 48.1%, respectively. The benzene selectivity with catalyst “A” at WSHV of 4.0 and 6.0 was 41.6%, and 41.5%, respectively. In contrast, the benzene selectivity with catalyst “X” at WSHV of 4.0 and 6.0 was 40.3%, and 40.5%, respectively. At both space velocities, the conversion of toluene, the selectivity for xylene isomers, and the selectivity for benzene were higher than that of catalyst “X,” which lacks macropores. Based on first order, reversible equilibrium reaction kinetics, the relative activity of catalyst “A” is 1.38 times that of catalyst “X,” which lacks macropores.

### Example 3

This example illustrates the performance capabilities of another catalyst containing macropores, catalyst “B,” to convert nitration-grade toluene to a product comprising xylene isomers. Separate runs were conducted with nearly-identical feeds at a WHSV of 4.0 and at a WHSV of 6.0. The feed stream was a mixture of hydrogen and toluene (4:1 hydrogen:toluene molar ratio), and the reactor conditions were those as set out above. Analyses of the liquid feed (feed. Wt. %) and products (Pdt. Wt. %) obtained in each run are shown in Table 4, below.

TABLE 4
Catalyst “B”

| | Feed | Product | Feed | Product |
|---|---|---|---|---|
| WHSV | 4.0 | | 6.0 | |
| Non-aromatics | 0.06 | 1.77 | 0.06 | 1.33 |
| Benzene | 0.00 | 19.57 | 0.00 | 16.49 |
| Toluene | 99.87 | 53.43 | 99.85 | 60.70 |
| Ethylbenzene | 0.03 | 0.17 | 0.03 | 0.14 |
| p-Xylene | 0.01 | 5.17 | 0.01 | 4.54 |
| m-Xylene | 0.02 | 11.25 | 0.02 | 9.96 |
| o-Xylene | 0.00 | 4.97 | 0.00 | 4.30 |
| Propylbenzene | 0.00 | 0.00 | 0.00 | 0.00 |
| Methylethylbenzene | 0.00 | 0.22 | 0.00 | 0.21 |
| Trimethylbenzene | 0.00 | 3.16 | 0.00 | 2.24 |
| A₁₀₊ | 0.00 | 0.29 | 0.02 | 0.20 |

At WSHV of 4.0, there is 16.5% benzene and 18.7% xylene isomers (0.88 weight ratio) as the major products. At WSHV of 6.0, there is 19.6% benzene and 21.4% xylene isomers (0.92 weight ratio) as the major products. Based on the obtained data shown in Table 4, the toluene conversions at WSHV of 4.0 and 6.0 was 46.5%, and 39.2% respectively. In contrast, the toluene conversions of a similar feed at WSHV of 4.0 and 6.0 utilizing catalyst “X” was 39.0%, and 31.6%, respectively. At both space velocities, the conversion of toluene with catalyst “B” was higher than that obtained with catalyst “X,” which lacks macropores. Based on first order, reversible equilibrium reaction kinetics, the relative activity of catalyst “B” is 1.43 times that of catalyst “X,” which lacks macropores.

At both space velocities, the conversion of toluene with catalyst “B” was higher than that obtained with catalyst “X,” which lacks macropores. Based on first order, reversible equilibrium reaction kinetics, the relative activity of catalyst “B” is 1.43 times that of catalyst “X,” which lacks macropores.

The xylene isomers selectivity with catalyst “B” at WSHV of 4.0 and 6.0 was 46.0%, and 47.7%, respectively. As shown in Example 1, above, the xylene isomers selectivity with catalyst “X” at WSHV of 4.0 and 6.0 was 46.2%, and 48.1%, respectively.

This example demonstrates that a catalyst containing macropores has comparable selectivity, at higher conversions. Thus, the catalyst containing macropores provides significantly better activity without compromising selectivity for xylene isomers (and benzene). The benzene selectivity with catalyst “B” at WSHV of 4.0 and 6.0 was 42.1%, and 42.1%, respectively. In contrast, the benzene selectivity with catalyst “X” at WSHV of 4.0 and 6.0 was 40.3%, and 40.5%, respectively.

### Example 4

This example illustrates the performance capabilities of still another catalyst containing macropores, catalyst “C,” to convert nitration-grade toluene to a product comprising xylene isomers. Separate runs were conducted with nearly-identical feeds at a WHSV of 4.0 and at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and products (Pdt. Wt. %) obtained in each run are shown in Table 5, below.

TABLE 5
Catalyst “C”

| | Feed | Product | Feed | Product |
|---|---|---|---|---|
| WHSV | 4.0 | | 6.0 | |
| Non-aromatics | 0.21 | 1.77 | 0.16 | 1.25 |
| Benzene | 0.00 | 19.50 | 0.00 | 16.78 |
| Toluene | 99.68 | 54.00 | 99.77 | 60.60 |
| Ethylbenzene | 0.03 | 0.24 | 0.03 | 0.17 |
| p-Xylene | 0.01 | 5.07 | 0.01 | 4.50 |
| m-Xylene | 0.02 | 11.04 | 0.02 | 9.75 |
| o-Xylene | 0.00 | 4.84 | 0.00 | 4.26 |
| Propylbenzene | 0.00 | 0.01 | 0.00 | 0.01 |
| Methylethylbenzene | 0.00 | 0.33 | 0.00 | 0.27 |
| Trimethylbenzene | 0.00 | 3.01 | 0.00 | 2.25 |
| A₁₀₊ | 0.04 | 0.24 | 0.01 | 0.18 |

At WSHV of 4.0, there is 19.5% benzene and 21.0% xylene isomers (0.93 weight ratio) as the major products. At WSHV of 6.0, there is 16.8% benzene and 18.5% xylene isomers (0.91 weight ratio) as the major products. Based on the obtained data shown in Table 5, the toluene conversions at WSHV of 4.0 and 6.0 was 46.5%, and 39.2% respectively. In contrast, the toluene conversions of a similar feed at WSHV of 4.0 and 6.0 utilizing catalyst “X” was 39.0%, and 31.6%, respectively. At both space velocities, the conversion of toluene was higher than that obtained with catalyst “X,” which lacks macropores. Based on first order, reversible equilibrium reaction kinetics, the relative activity of catalyst “C” is 1.42 times that of catalyst “X,” which lacks macropores.

The xylene isomers selectivity with catalyst “C” at WSHV of 4.0 and 6.0 was 45.7%, and 47.2%, respectively. As shown in Example 1, above, the xylene isomers selectivity with catalyst “X” at WSHV of 4.0 and 6.0 was 46.2%, and 48.1%, respectively. The benzene selectivity with catalyst “C” at WSHV of 4.0 and 6.0 was 42.6%, and 42.7%, respectively. In contrast, the benzene selectivity with catalyst “X” at WSHV of 4.0 and 6.0 was 40.3%, and 40.5%, respectively. This example demonstrates that a catalyst containing macropores has comparable selectivity, at higher conversions. Thus, the catalyst containing macropores provides significantly better activity without compromising selectivity for xylene isomers (and benzene).

### Example 5

This example illustrates the performance capabilities of yet another catalyst containing macropores, catalyst “D,” to convert nitration-grade toluene to a product comprising xylene isomers. One run was conducted with the feed at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and product (Pdt. Wt. %) obtained in the run are shown in Table 6, below.

TABLE 6
Catalyst “D”

| | Feed | Product |
|---|---|---|
| WHSV | 6.0 | |
| Non-aromatics | 0.07 | 1.19 |
| Benzene | 0.00 | 15.51 |
| Toluene | 99.68 | 62.29 |
| Ethylbenzene | 0.03 | 0.19 |
| p-Xylene | 0.01 | 4.42 |
| m-Xylene | 0.02 | 9.56 |
| o-Xylene | 0.13 | 4.18 |
| Propylbenzene | 0.00 | 0.01 |
| Methylethylbenzene | 0.00 | 0.34 |
| Trimethylbenzene | 0.00 | 2.12 |
| A₁₀₊ | 0.05 | 0.19 |

At WSHV of 6.0, there is 15.5% benzene and 18.2% xylene isomers (0.85 weight ratio) as the major products. Based on the obtained data shown in Table 6, the toluene conversion at WSHV of 6.0 was 37.5%. In contrast, the toluene conversions of a similar feed at WSHV of 6.0 utilizing catalyst “X” was 31.6%. The conversion of toluene was higher than that obtained with catalyst “X,” which lacks macropores. Based on first order, reversible equilibrium reaction kinetics, the relative activity of catalyst “D” is 1.38 times that of catalyst “X,” which lacks macropores.

The xylene isomers selectivity with catalyst “D” at WSHV of 6.0 was 48.4%. In contrast, the xylene isomers selectivity with catalyst “X” at WSHV of 6.0 was 48.1%. The benzene selectivity with catalyst “D” at WSHV of 6.0 was 41.4%. As shown in Example 1, above, the benzene selectivity with catalyst “X” at WSHV of 6.0 was 40.5%. This example demonstrates that a catalyst containing macropores has comparable selectivity, at higher conversions. Thus, the catalyst containing macropores provides significantly better activity without compromising selectivity for xylene isomers (and benzene).

### Example 6

This example illustrates the performance capabilities of yet another catalyst containing macropores, catalyst “E,” to convert nitration-grade toluene to a product comprising xylene isomers. One run was conducted with the feed at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and product (Pdt. Wt. %) obtained in the run are shown in Table 7, below.

TABLE 7
Catalyst “E”

| | Feed | Product |
|---|---|---|
| WHSV | 6.0 | |
| Non-aromatics | 0.25 | 0.85 |
| Benzene | 0.00 | 13.69 |
| Toluene | 99.68 | 67.69 |
| Ethylbenzene | 0.03 | 0.14 |
| p-Xylene | 0.01 | 3.83 |
| m-Xylene | 0.03 | 8.24 |
| o-Xylene | 0.00 | 3.60 |
| Propylbenzene | 0.00 | 0.01 |
| Methylethylbenzene | 0.00 | 0.28 |
| Trimethylbenzene | 0.00 | 1.55 |
| A₁₀₊ | 0.00 | 0.12 |

The conversion of toluene was higher than that obtained with catalyst “X,” which lacks macropores. Based on first order, reversible equilibrium reaction kinetics, the relative activity of catalyst “E” is 1.15 times that of catalyst “X,” which lacks macropores. Catalyst “E” exhibits improved activity relative to catalyst “X” even though catalyst “E” contains only 70% H-mordenite compared to catalyst “X,” which has 80% H-mordenite. At WSHV of 6.0, there is 13.7% benzene and 15.7% xylene isomers (0.87 weight ratio) as the major products. Based on the obtained data shown in Table 7, the toluene conversion at WSHV of 6.0 was 32.1%. In contrast, the toluene conversions of a similar feed at WSHV of 6.0 utilizing catalyst “X” was 31.6%.

The xylene isomers selectivity with catalyst “E” at WSHV of 6.0 was 42.8%. In contrast, the xylene isomers selectivity with catalyst “X” at WSHV of 6.0 was 48.1%. The benzene selectivity with catalyst “E” at WSHV of 6.0 was 42.7%. As shown in Example 1, above, the benzene selectivity with catalyst “X” at WSHV of 6.0 was 40.5%. This example demonstrates that a catalyst containing macropores has comparable selectivity, at higher conversions. Thus, the catalyst containing macropores provides significantly better activity without compromising selectivity for xylene isomers (and benzene).

### Example 7

This example illustrates the performance capabilities of yet another catalyst containing macropores, catalyst “F,” to convert nitration-grade toluene to a product comprising xylene isomers. One run was conducted with the feed at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and product (Pdt. Wt. %) obtained in the run are shown in Table 8, below.

TABLE 8
Catalyst “F”

| | Feed | Product |
|---|---|---|
| WHSV | 6.0 | |
| Non-aromatics | 0.07 | 1.15 |
| Benzene | 0.00 | 16.36 |
| Toluene | 99.70 | 61.76 |
| Ethylbenzene | 0.03 | 0.20 |
| p-Xylene | 0.01 | 4.36 |
| m-Xylene | 0.02 | 9.42 |
| o-Xylene | 0.16 | 4.12 |
| Propylbenzene | 0.00 | 0.01 |
| Methylethylbenzene | 0.00 | 0.34 |
| Trimethylbenzene | 0.00 | 2.13 |
| A₁₀₊ | 0.00 | 0.16 |

At WSHV of 6.0, there is 16.4% benzene and 17.9% xylene isomers (0.92 weight ratio) as the major products. Based on the obtained data shown in Table 8, the toluene conversion at WSHV of 6.0 was 38.1%. In contrast, the toluene conversions of a similar feed at WSHV of 6.0 utilizing catalyst “X” was 31.6%. The conversion of toluene was higher than that obtained with catalyst “X,” which lacks macropores. Based on first order, reversible equilibrium reaction kinetics, the relative activity of catalyst “F” is 1.38 times that of catalyst “X,” which lacks macropores.

The xylene isomers selectivity with catalyst “F” at WSHV of 6.0 was 47.0%. In contrast, the xylene isomers selectivity with catalyst “X” at WSHV of 6.0 was 48.1%. The benzene selectivity with catalyst “F” at WSHV of 6.0 was 43.0%. In contrast, the benzene selectivity with catalyst “X” at WSHV of 6.0 was 40.5%.

### Example 8

This example illustrates the performance capabilities of another catalyst containing macropores, catalyst “G,” to convert nitration-grade toluene to a product comprising xylene isomers. Separate runs were conducted with nearly-identical feeds at a WHSV of 4.0 and at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and products (Pdt. Wt. %) obtained in each run are shown in Table 9, below.

TABLE 9
Catalyst “G”

| | Feed | Product | Feed | Product |
|---|---|---|---|---|
| WHSV | 4.0 | | 6.0 | |
| Non-aromatics | 0.08 | 1.74 | 0.08 | 1.17 |
| Benzene | 0.00 | 20.00 | 0.00 | 16.95 |
| Toluene | 99.64 | 52.69 | 99.63 | 59.37 |
| Ethylbenzene | 0.03 | 0.31 | 0.03 | 0.22 |
| p-Xylene | 0.01 | 5.17 | 0.01 | 4.69 |
| m-Xylene | 0.02 | 11.25 | 0.02 | 10.17 |
| o-Xylene | 0.17 | 4.95 | 0.16 | 4.46 |
| Propylbenzene | 0.00 | 0.01 | 0.00 | 0.01 |
| Methylethylbenzene | 0.00 | 0.43 | 0.00 | 0.38 |
| Trimethylbenzene | 0.00 | 3.14 | 0.00 | 2.35 |
| A₁₀₊ | 0.05 | 0.32 | 0.06 | 0.23 |

At WSHV of 4.0, there is 20.0% benzene and 21.4% xylene isomers (0.93 weight ratio) as the major products. At WSHV of 6.0, there is 17.0% benzene and 19.3% xylene isomers (0.88 weight ratio) as the major products. Based on the obtained data shown in Table 9, the toluene conversions at WSHV of 4.0 and 6.0 was 47%, and 40% respectively. In contrast, the toluene conversions of a similar feed at WSHV of 4.0 and 6.0 utilizing catalyst “X” was 39.0%, and 31.6%, respectively. At both space velocities, the conversion of toluene was higher than that of catalyst “X,” which lacks macropores. Based on first order, reversible equilibrium reaction kinetics, the relative activity of catalyst “G” is 1.48 times that of catalyst “X,” which lacks macropores.

The xylene isomers selectivity with catalyst “G” at WSHV of 4.0 and 6.0 was 45.5%, and 48.3%, respectively. As shown in Example 1, above, the xylene isomers selectivity with catalyst “X” at WSHV of 4.0 and 6.0 was 46.2%, and 48.1%, respectively. The benzene selectivity with catalyst “G” at WSHV of 4.0 and 6.0 was 42.5%, and 42.4%, respectively. As shown in Example 1, above, the benzene selectivity with catalyst “X” at WSHV of 4.0 and 6.0 was 40.3%, and 40.5%, respectively. This example demonstrates that a catalyst containing macropores has comparable selectivity, at higher conversions. Thus, the catalyst containing macropores provides significantly better activity without compromising selectivity for xylene isomers (and benzene).

Table 10, below, summarizes the catalyst properties and relative activities of catalysts “X” and “A” through “G.” As noted above, all catalysts “X” and “A” through “G” contained 80% H-mordenite, except that catalyst “E” contained only 70% H-mordenite notwithstanding, and as below, catalyst “E” provides improved activity relative to catalyst “X”.

TABLE 10

| | | Relative | Hg Macropore Vol. |
|---|---|---|---|
| Catalyst | Size & Shape | Activity | (50-100 nm) (cc/g) |
| X | 1/12″ cylinder | 1.00 | 0.018 |
| A | 1/16″ Trilobe | 1.38 | 0.132 |
| B | 1/12″ cylinder | 1.43 | 0.255 |
| C | 1/12″ cylinder | 1.42 | 0.288 |
| D | 1/12″ cylinder | 1.38 | 0.280 |
| E | 1/12″ cylinder | 1.15 | 0.212 |
| F | 1/12″ cylinder | 1.38 | 0.281 |
| G | 1/16″ Trilobe | 1.48 | 0.280 |

For the larger, 1/12-inch cylindrical extrudates greater than about 0.2 cc/g is required to give the highest activity. For example, there is a benefit to the activity (1.15 times higher) by increasing the macropore volume to 0.212 cc/g (catalyst “E”) compared to catalyst “X.” However, for catalysts with macropore volume greater than 0.25 cc/g, the activity increases to a maximum of near 1.4 (catalysts “B”, “C”, “D,” and “F”). For smaller size extrudates lower amounts of macropore volume can give acceptable activity. For example, if the extrudate size is reduced to 1/16 inch and the shape is chosen to give an even smaller effective size (catalyst “A”), a catalyst with a maximum activity near 1.4 is achieved with a macropore volume of 0.13 cc/g. The amount of macropore volume, therefore, depends on the size of the extrudate and values from about 0.1 to 03 cc/g are most preferred. Macropore volumes above about 0.02 cc/g, however, lead to catalysts with higher relative activity.

### Example 9

This example compares the performance capabilities of a catalyst lacking macropores (catalyst “H”) versus a catalyst containing macropores (catalyst “I”) to convert nitration-grade toluene to a product comprising xylene isomers. Separate runs with each catalyst were conducted with nearly-identical feeds at a WHSV of 6.0. The feed stream was a mixture of hydrogen and toluene (4:1 hydrogen:toluene molar ratio), and the reactor conditions were those as set out above. Analyses of the liquid feed (Feed. Wt. %) and products (Pdt. Wt. %) obtained with each catalyst on consecutive days, and analyses of the conversion are presented in Table 11 (catalyst “H”) and Table 12 (catalyst “I”), below:

TABLE 11
Catalyst “H”

| | | Product | Product |
|---|---|---|---|
| | Feed | Day 1 | Day 2 |
| Non-aromatics | 0.09 | 1.48 | 1.21 |
| Benzene | 0.00 | 11.92 | 9.90 |
| Toluene | 99.83 | 70.69 | 75.83 |
| Ethylbenzene (EB) | 0.04 | 0.12 | 0.09 |
| p-Xylene | 0.01 | 3.50 | 2.91 |
| m-Xylene | 0.02 | 7.50 | 6.21 |
| o-Xylene | 0.00 | 3.25 | 2.71 |
| Propylbenzene | 0.00 | 0.00 | 0.00 |
| Methylethylbenzene | 0.00 | 0.26 | 0.23 |
| Trimethylbenzene | 0.00 | 1.11 | 0.78 |
| A₁₀₊ | 0.00 | 1.17 | 0.13 |

| | | |
|---|---|---|
| Benzene/Xylenes | 0.84 | 0.84 |
| Toluene conversion | 29.19 | 24.04 |
| EB selectivity in C₈ fraction | 0.84 | 0.75 |
| Selectivity to C₉ aromatics | 5.29 | 4.73 |

TABLE 12
Catalyst “I”

| | | Product | Product | Product |
|---|---|---|---|---|
| | Feed | Day 1 | Day 2 | Day 3 |
| Non-aromatics | 0.09 | 0.70 | 0.70 | 0.70 |
| Benzene | 0.02 | 18.55 | 19.20 | 19.40 |
| Toluene | 99.79 | 55.50 | 55.01 | 55.10 |
| Ethylbenzene (EB) | 0.04 | 0.19 | 0.17 | 0.17 |
| p-Xylene | 0.02 | 5.24 | 5.21 | 5.16 |
| m-Xylene | 0.04 | 11.35 | 11.29 | 11.18 |
| o-Xylene | 0.00 | 4.95 | 4.93 | 4.88 |
| Propylbenzene | 0.00 | 0.01 | 0.01 | 0.00 |
| Methylethylbenzene | 0.01 | 0.27 | 0.24 | 0.23 |
| Trimethylbenzene | 0.01 | 3.01 | 3.02 | 2.96 |
| A₁₀₊ | 0.00 | 0.23 | 0.22 | 0.21 |

| | | | |
|---|---|---|---|
| Benzene/Xylenes | 0.86 | 0.90 | 0.92 |
| Toluene conversion | 44.37 | 44.86 | 44.76 |
| EB selectivity in C₈ fraction | 0.90 | 0.81 | 0.78 |
| Selectivity to C₉ aromatics | 7.90 | 7.74 | 7.57 |

Based on the foregoing data, the catalyst containing no macropore volume, catalyst “H,” had low toluene conversion (24% on day 1 and 29% on day 2) when compared to the toluene conversion achieved with catalyst “I” (consistently about 44% on each day) which contains macropore volume. Additionally, it was observed that the catalyst “1” provided stable performance (i.e., no loss of activity), while catalyst “H” did not provide equally stable performance, losing 5% toluene conversion in 1-2 days. Thus, the foregoing example demonstrates that a catalyst containing macropore volume is more stable than a catalyst lacking macropore volume.

### Example 10

This example demonstrates the ability of catalyst “C” (a macroporous catalyst) to convert a feed containing toluene, benzene, and some light non-aromatics to xylene isomers. Three nearly identical feeds were converted by the catalyst. In the three runs, the reaction conditions were identical except that the temperature of the reactor and the WHSV were different. Analyses of the liquid feed (Feed. Wt. %), obtained product (Pdt. Wt. %), and the conversion are presented in Table 13, below.

TABLE 13
Catalyst “C”

| | Feed | Pdt. | Feed | Pdt. | Feed | Pdt. |
|---|---|---|---|---|---|---|
| WHSV | 0.5 | | 2.0 | | 3.0 | |
| Non-aromatics | 3.05 | 3.58 | 3.61 | 6.04 | 3.05 | 3.84 |
| Benzene | 7.59 | 21.71 | 5.91 | 20.72 | 7.64 | 19.86 |
| Toluene | 82.89 | 49.38 | 79.76 | 45.43 | 82.85 | 55.09 |
| Ethylbenzene | 1.00 | 0.84 | 2.07 | 0.68 | 1.00 | 0.52 |
| p-Xylene | 1.77 | 4.90 | 2.88 | 5.32 | 1.77 | 4.30 |
| m-Xylene | 3.29 | 10.78 | 5.26 | 11.68 | 3.28 | 9.35 |
| o-Xylene | 0.42 | 4.48 | 0.52 | 5.02 | 0.41 | 4.08 |
| Propylbenzene | 0.00 | 0.02 | 0.00 | 0.01 | 0.00 | 0.01 |
| Methylethylbenzene | 0.00 | 0.96 | 0.00 | 0.74 | 0.00 | 0.59 |
| Trimethylbenzene | 0.00 | 2.94 | 0.00 | 3.80 | 0.00 | 2.14 |
| A₁₀₊ | 0.00 | 0.42 | 0.00 | 0.56 | 0.00 | 0.22 |

| | | | |
|---|---|---|---|
| Reactor pressure (psig) | 200 | 200 | 200 |
| Reactor temperature (° F.) | 750 | 720 | 750 |
| Benzene/Xylenes | 0.96 | 1.11 | 1.00 |
| Toluene conversion | 40.43 | 43.04 | 33.50 |
| EB selectivity in C₈ fraction | 3.98 | 3.00 | 2.84 |
| Selectivity to C₉ aromatics | 10.73 | 11.88 | 8.84 |

Where the WHSV was 0.5, the net benzene obtained in the product of the conversion was 14.12, whereas the net xylene isomers obtained in the product of the conversion was 14.68. Accordingly, the ratio of net benzene to net xylene isomers (Benzene/Xylenes) obtained by converting this toluene feed is 0.96 (i.e., 0.96=(14.12+14.68)). This ratio is reported in the foregoing table as Benzene/Xylenes. The data in the foregoing table also show that, when the temperature and pressure remain constant (750° F. and 200 psig) and the WHSV is increased (from 0.5 to 3.0), toluene conversion, ethylbenzene selectivity in the C₈ fraction, and selectivity to C₉ aromatics decrease. The decrease in toluene conversion as WHSV is increased is an expected response because as more feed is passed over a catalyst over a given time, more demands are placed on the catalyst resulting in less conversion. Generally, toluene conversion will depend upon the temperature, pressure, and WHSV, the WHSV being a combination of the amount of catalyst and the feed rate.

This example also demonstrates the subject catalyst is capable of converting the feed, producing a net increase in xylene isomers, and ensuring a net decrease in ethylbenzene. While not wishing to be bound to any particular theory, it is believed that the ethyl group on the ethylbenzene is removed by the catalyst and then saturated by the hydrogenation component of the catalyst (molybdenum in this catalyst) to form ethane, which is non-reactive. The absence of the hydrogenation component would likely leave a reactive ethyl group in the product mixture, which could undesirably react with a desirable component in the mixture.

Typically, a person having ordinary skill in the art would not attempt (or expect) to convert non-aromatics (e.g., paraffins) with a conventional catalyst because such feeds would be highly detrimental to the catalyst. Specifically, non-aromatics will react when exposed to a conventional catalyst yielding products that will rapidly deactivate the catalyst and change the selectivity of the catalyst. Instead, the skilled artisan would pass such feeds through expensive unit operations to extract the non-aromatics from the feed before attempting to convert the feed.

The foregoing example demonstrates that the disclosed macroporous catalyst can be used to convert a feed containing at least about 3 wt. % non-aromatics without suffering from the disadvantages prevalent with conventional catalysts. Moreover, the ability to convert such feeds obviates the necessity to extract non-aromatics in advance of the conversion, which imparts a significant operations cost savings.

It has also been discovered that the benzene produced by the conversion with this molybdenum-impregnated macroporous catalyst has a purity acceptable to the refining industry (i.e., less than 0.1% of the benzene is actually saturated). This is an unexpected benefit. Although such purity might be obtainable using a non-metal impregnated catalyst, the toluene feed must not contain non-aromatics. If non-aromatics are present, then the skilled artisan would impregnate the catalyst with platinum or nickel. In no doing, the benzene in the product of the conversion would be unacceptable to the refining industry, which requires at least 99.9% benzene purity.

### Example 11

This example demonstrates the ability of catalyst “G” (a macroporous catalyst) to convert a feed containing toluene, benzene, and some light non-aromatics to xylene isomers. Analyses of the liquid feed (Feed. Wt. %), obtained product (Pdt. Wt %), and the conversion are presented in Table 14, below.

TABLE 14
Catalyst “G”

| | Feed | Pdt. |
|---|---|---|
| WHSV | 5.9 | |
| Non-aromatics | 3.76 | 4.88 |
| Benzene | 5.40 | 18.77 |
| Toluene | 79.52 | 50.32 |
| Ethylbenzene | 2.19 | 0.61 |
| p-Xylene | 3.03 | 5.15 |
| m-Xylene | 5.52 | 11.18 |
| o-Xylene | 0.55 | 4.95 |
| Propylbenzene | 0.00 | 0.01 |
| Methylethylbenzene | 0.00 | 0.66 |
| Trimethylbenzene | 0.01 | 3.14 |
| A₁₀₊ | 0.02 | 0.33 |

| | |
|---|---|
| Reactor pressure (psig) | 200 |
| Reactor temperature (° F.) | 780 |
| Benzene/Xylenes | 1.10 |
| Toluene conversion | 36.73 |
| EB selectivity in C₈ fraction | 2.79 |
| Selectivity to C₉ aromatics | 11.21 |

Examples 2 through 8, above, demonstrated that a macroporous catalyst impregnated with molybdenum can be used to convert nitration grade toluene, while Example 10 and this example demonstrate the ability of such a catalyst to convert a toluene feed containing previously-undesired non-aromatics. The processing flexibility afforded by the catalyst and this method is a great benefit to the engineer because it obviates the need for complicated process modifications that are dependent upon the precise composition of the toluene feed, and produces xylene isomers as well as useable benzene.

### Example 12

This example demonstrates the ability of catalyst “C” (a macroporous catalyst) to convert a feed containing C₉₊ aromatics to xylene isomers. Analyses of the liquid feed (Feed. Wt. %), obtained product (Pdt. Wt. %), and the conversion are presented in Table 15, below.

TABLE 15
Catalyst “C”

| | Feed | Pdt. |
|---|---|---|
| WHSV | 1.0 | |
| Non-aromatics | 0.85 | 20.59 |
| Benzene | 0.09 | 4.45 |
| Toluene | 0.24 | 16.17 |
| Ethylbenzene | 0.00 | 5.95 |
| p-Xylene | 0.01 | 4.98 |
| m-Xylene | 0.02 | 10.97 |
| o-Xylene | 0.50 | 4.65 |
| Propylbenzene | 0.17 | 0.00 |
| Methylethylbenzene | 17.16 | 10.64 |
| Trimethylbenzene | 18.24 | 9.75 |
| A₁₀₊ | 62.73 | 11.83 |

| | |
|---|---|
| Reactor pressure (psig) | 200 |
| Reactor temperature (° F.) | 680 |
| Benzene/Xylenes | 0.22 |
| Toluene Yield | 15.94 |
| EB selectivity in C₈ fraction | 22.40 |
| C₉₊ aromatics conversion | 67.20 |

This example demonstrates that the method can convert a feed containing C₉₊ aromatics. Heretofore, a person skilled in the art would not attempt (or expect) to convert the feed with a conventional catalyst because the C₁₀₊ aromatics would rapidly deactivate the catalyst. Thus, the skilled artisan would fractionate the feed to remove C₁₀₊ aromatics before attempting the conversion with the conventional catalyst. This example, however, demonstrates that the macroporous catalyst impregnated with a hydrogenation component can be used to convert a feed containing C₉₊ aromatics, thereby advantageously obviating a fractionation step.

This is important because C₁₀₊ aromatics are often present in the product of a conversion. See e.g., Examples 1 through 11, above. As noted above, such a product could not undergo further conversion with a conventional catalyst due to the catalyst deactivation effect of the C₁₀₊ aromatics. However, because such deactivation is not a problem with the catalyst disclosed herein, and because such C₁₀₊ aromatics can be converted by the catalyst, feeds containing C₁₀₊ aromatics can be recycled with fresh feed without the necessity of fractionating the recycle to remove such C₁₀₊ aromatics.

### Example 13

This example demonstrates the ability of catalyst “G” (a macroporous catalyst) to convert a feed containing C₉₊ aromatics to xylene isomers. Five nearly identical feeds were converted by the catalyst, in the five runs, the reaction conditions were identical except that the temperature of the reactor was changed in each run. Analyses of the liquid feed (Feed. Wt. %), obtained product (Pdt. Wt. %), and the conversion are presented in Table 16, below.

TABLE 16
Catalyst “G”

| | Feed | Pdt. | Feed | Pdt. | Feed | Pdt. | Feed | Pdt. | Feed | Pdt. |
|---|---|---|---|---|---|---|---|---|---|---|
| WHSV | 1.0 | | 1.0 | | 1.0 | | 1.0 | | 1.0 | |
| Non-aromatics | 0.89 | 20.57 | 0.86 | 23.57 | 0.84 | 27.37 | 0.83 | 29.17 | 0.85 | 28.86 |
| Benzene | 0.05 | 4.74 | 0.05 | 6.28 | 0.05 | 7.99 | 0.05 | 8.52 | 0.05 | 8.71 |
| Toluene | 0.08 | 17.21 | 0.09 | 21.78 | 0.03 | 25.10 | 0.03 | 25.76 | 0.03 | 25.68 |
| Ethylbenzene | 0.00 | 6.28 | 0.00 | 3.78 | 0.00 | 0.92 | 0.00 | 0.40 | 0.00 | 0.45 |
| p-Xylene | 0.01 | 5.26 | 0.01 | 5.86 | 0.00 | 6.15 | 0.00 | 6.06 | 0.00 | 6.03 |
| m-Xylene | 0.02 | 11.59 | 0.02 | 12.91 | 0.02 | 13.50 | 0.01 | 13.27 | 0.02 | 13.19 |
| o-Xylene | 0.49 | 4.91 | 0.50 | 5.54 | 0.49 | 5.95 | 0.49 | 5.89 | 0.49 | 5.88 |
| Propylbenzene | 0.17 | 0.00 | 0.17 | 0.00 | 0.17 | 0.00 | 0.17 | 0.00 | 0.17 | 0.01 |
| Methylethylbenzene | 17.14 | 10.35 | 17.22 | 5.99 | 17.09 | 1.36 | 16.99 | 0.57 | 17.00 | 0.54 |
| Trimethylbenzene | 18.22 | 9.90 | 18.29 | 9.73 | 18.16 | 9.46 | 18.09 | 8.99 | 18.09 | 9.08 |
| A₁₀₊ | 62.95 | 9.20 | 62.80 | 4.55 | 63.14 | 2.18 | 63.33 | 1.36 | 63.31 | 1.57 |

| | | | | | |
|---|---|---|---|---|---|
| Reactor pressure (psig) | 200 | 200 | 200 | 200 | 200 |
| Reactor temperature (° F.) | 675 | 700 | 750 | 775 | 780 |
| Benzene/Xylenes | 0.22 | 0.26 | 0.32 | 0.34 | 0.35 |
| Toluene Yield | 17.13 | 21.69 | 25.07 | 25.72 | 25.65 |
| EB selectivity in C₈ fraction | 22.39 | 13.45 | 3.47 | 1.56 | 1.75 |
| C₉₊ aromatics conversion | 70.09 | 79.41 | 86.80 | 88.91 | 88.64 |

### Example 14

This example demonstrates the ability of catalyst “D” (a macroporous catalyst) to convert a feed containing C₉₊ aromatics (and predominantly C₉ aromatics) to xylene isomers. Analyses of the liquid feed (Feed. Wt. %), obtained product (Pdt. Wt. %), and the conversion are presented in Table 17, below.

TABLE 17
Catalyst “D”

| | Feed | Pdt. |
|---|---|---|
| WHSV | 2.0 | |
| Non-aromatics | 0.20 | 12.21 |
| Benzene | 0.12 | 4.74 |
| Toluene | 0.07 | 23.06 |
| Ethylbenzene | 0.00 | 0.62 |
| p-Xylene | 0.01 | 8.45 |
| m-Xylene | 0.02 | 18.48 |
| o-Xylene | 0.10 | 8.11 |
| Propylbenzene | 6.67 | 0.00 |
| Methylethylbenzene | 49.66 | 1.46 |
| Trimethylbenzene | 42.28 | 19.05 |
| A₁₀₊ | 0.88 | 3.83 |

| | |
|---|---|
| Reactor pressure (psig) | 200 |
| Reactor temperature (° F.) | 750 |
| Benzene/Xylenes | 0.13 |
| Toluene Yield | 22.99 |
| EB selectivity in C₈ fraction | 1.75 |
| C₉₊ aromatics conversion | 75.54 |

### Example 15

This example demonstrates the ability of catalyst “C” (a macroporous catalyst) to convert a feed containing C₉₊ aromatics (and predominantly C₉ aromatics) to xylene isomers. Six nearly identical feeds were converted by the catalyst. In the six runs, the reaction conditions were identical except that the temperature of the reactor was changed in each run. Analyses of the liquid feed (Feed. Wt. %), obtained product (Pdt. Wt. %), and the conversion are presented in Table 18, below.

Where the produced xylene isomers will undergo downstream conversion operations to produce para-xylenes, ethylbenzene present in the C₈ aromatics fraction must be converted to benzene by a dealkylation (de-ethylation) process. Such de-ethylation requires passing the fraction over another catalyst to remove the ethyl group from the ethylbenzene. This de-ethylation can destroy some of the xylene isomers present in the C₈ aromatics fraction, ultimately resulting in yield losses of xylene isomers. Based on the data below, low ethylbenzene selectivity in the C₈ aromatics fraction is achievable by the demonstrated method. Such low ethylbenzene is desirable because it reduces the expense in downstream processing and improved yield of xylene recovery in para-xylene processino units.

TABLE 18
Catalyst “C”

| | Feed | Pdt. | Feed | Pdt. | Feed | Pdt. | Feed | Pdt. | Feed | Pdt. | Feed | Pdt. |
|---|---|---|---|---|---|---|---|---|---|---|---|---|
| WHSV | 1.0 | | 1.0 | | 1.0 | | 1.0 | | 1.0 | | 1.0 | |
| Non-aromatics | 0.19 | 4.00 | 0.19 | 3.98 | 0.19 | 11.72 | 0.20 | 18.33 | 0.21 | 15.71 | 0.20 | 15.90 |
| Benzene | 0.12 | 1.93 | 0.12 | 2.67 | 0.12 | 3.43 | 0.12 | 3.98 | 0.12 | 4.53 | 0.12 | 4.66 |
| Toluene | 0.02 | 12.97 | 0.02 | 16.43 | 0.02 | 18.97 | 0.02 | 20.00 | 0.03 | 22.11 | 0.03 | 22.40 |
| Ethylbenzene | 0.01 | 4.01 | 0.01 | 3.62 | 0.00 | 2.10 | 0.00 | 1.06 | 0.00 | 0.54 | 0.01 | 0.42 |
| p-Xylene | 0.02 | 5.22 | 0.02 | 6.75 | 0.00 | 7.57 | 0.00 | 7.59 | 0.00 | 8.11 | 0.02 | 8.13 |
| m-Xylene | 0.06 | 11.44 | 0.06 | 14.84 | 0.01 | 16.65 | 0.01 | 16.66 | 0.01 | 17.79 | 0.06 | 17.79 |
| o-Xylene | 0.12 | 4.85 | 0.12 | 6.28 | 0.09 | 7.14 | 0.09 | 7.22 | 0.09 | 7.80 | 0.12 | 7.85 |
| Propylbenzene | 6.65 | 0.03 | 6.65 | 0.00 | 6.65 | 0.00 | 6.66 | 0.00 | 6.66 | 0.00 | 6.65 | 0.00 |
| Methylethylbenzene | 49.63 | 16.00 | 49.62 | 11.33 | 49.66 | 5.60 | 49.67 | 2.59 | 49.64 | 1.29 | 49.60 | 0.96 |
| Trimethylbenzene | 42.38 | 23.00 | 42.37 | 21.51 | 42.41 | 19.47 | 42.49 | 17.95 | 42.48 | 18.44 | 42.40 | 18.42 |
| A₁₀₊ | 0.82 | 16.55 | 0.84 | 12.59 | 0.84 | 7.35 | 0.75 | 4.62 | 0.76 | 3.68 | 0.81 | 3.46 |

| | | | | | | |
|---|---|---|---|---|---|---|
| Reactor pressure (psig) | 200 | 200 | 200 | 200 | 200 | 200 |
| Reactor temperature (° F.) | 650 | 675 | 700 | 725 | 750 | 775 |
| Benzene/Xylenes | 0.09 | 0.09 | 0.11 | 0.12 | 0.13 | 0.14 |
| Toluene Yield | 12.95 | 16.41 | 18.95 | 19.98 | 22.08 | 22.37 |
| EB selectivity in C₈ fraction | 15.71 | 11.48 | 6.27 | 3.24 | 1.59 | 1.24 |
| C₉₊ aromatics conversion | 44.12 | 54.33 | 67.44 | 74.73 | 76.47 | 77.02 |

Based on the foregoing data, at constant pressure and WHSV, toluene yield and the conversion of C₉₊ aromatics increase as the temperature increases. Similarly, at constant pressure and WHSV, ethylbenzene selectivity in the C₈ aromatics fraction decreases as the temperature increases.

### Example 16

This example demonstrates the ability of catalyst “D” (a macroporous catalyst) to convert a feed containing C₉ aromatics and toluene to xylene isomers. Analyses of the liquid feed (Feed. Wt. %), obtained product (Pdt. Wt. %), and the conversion are presented in Table 19, below.

TABLE 19
Catalyst “D”

| | Feed | Pdt. |
|---|---|---|
| WHSV | 1.0 | |
| Non-aromatics | 0.19 | 6.53 |
| Benzene | 0.01 | 9.50 |
| Toluene | 49.29 | 36.55 |
| Ethylbenzene | 0.02 | 2.09 |
| p-Xylene | 0.02 | 7.03 |
| m-Xylene | 0.04 | 15.36 |
| o-Xylene | 0.06 | 6.78 |
| Propylbenzene | 3.33 | 0.00 |
| Methylethylbenzene | 25.04 | 3.78 |
| Trimethylbenzene | 21.50 | 10.03 |
| A₁₀₊ | 0.50 | 2.34 |

| | |
|---|---|
| Reactor pressure (psig) | 200 |
| Reactor temperature (° F.) | 680 |
| Benzene/Xylenes | 0.33 |
| Toluene Conversion | 25.85 |
| EB selectivity in C₈ fraction | 6.69 |
| C₉₊ aromatics conversion | 67.92 |

This example also demonstrates the production of C₉₊ aromatics, which in accordance with the prior examples (e.g., Examples 12 and 13), can be recycled back to the feed for further conversion with the same type of catalyst. This example further demonstrates the flexibility of the method to accommodate multiple feed operations utilizing the same general process configuration, removing products of the specific conversion as desired.

### Example 17

This example demonstrates the ability of catalyst “G” to convert a feed containing C₉₊ aromatics, benzene, and toluene to xylene isomers. Five nearly identical feeds were converted by the catalyst. Such a feed is representative of a feed containing recycle and, as explained above, the benefits of the method with use of a macroporous catalyst impregnated with a hydrogenation component include its ability to convert such feeds without requiring complicated and expensive upstream and downstream purification operations.

In the five runs, the reaction conditions were identical except that the temperature of the reactor was changed in each run. Analyses of the liquid feed (Feed. Wt %), obtained product (Pdt. Wt. %), and the conversion are presented in Table 20, below.

TABLE 20
Catalyst “G”

| | Feed | Pdt. | Feed | Pdt. | Feed | Pdt. | Feed | Pdt. | Feed | Pdt. |
|---|---|---|---|---|---|---|---|---|---|---|
| WHSV | 1.3 | | 1.3 | | 1.3 | | 1.3 | | 1.3 | |
| Non-aromatics | 2.14 | 10.72 | 2.17 | 13.15 | 2.14 | 14.97 | 2.15 | 16.61 | 2.58 | 17.35 |
| Benzene | 4.45 | 14.40 | 4.37 | 15.80 | 4.33 | 16.60 | 4.22 | 17.16 | 4.32 | 16.88 |
| Toluene | 49.07 | 35.96 | 48.85 | 36.44 | 48.93 | 36.07 | 48.61 | 35.84 | 50.75 | 35.52 |
| Ethylbenzene | 0.59 | 3.89 | 0.59 | 2.05 | 0.59 | 0.99 | 0.59 | 0.45 | 0.62 | 0.28 |
| p-Xylene | 1.04 | 5.60 | 1.05 | 5.73 | 1.05 | 5.74 | 1.05 | 5.62 | 1.10 | 5.63 |
| m-Xylene | 1.94 | 12.31 | 1.94 | 12.59 | 1.95 | 12.59 | 1.95 | 12.31 | 2.04 | 12.30 |
| o-Xylene | 0.45 | 5.26 | 0.45 | 5.43 | 0.45 | 5.52 | 0.45 | 5.44 | 0.47 | 5.49 |
| Propylbenzene | 0.09 | 0.00 | 0.09 | 0.01 | 0.09 | 0.01 | 0.09 | 0.01 | 0.09 | 0.01 |
| Methylethylbenzene | 7.19 | 4.50 | 7.23 | 2.19 | 7.22 | 1.04 | 7.27 | 0.47 | 7.59 | 0.30 |
| Trimethylbenzene | 7.57 | 5.43 | 7.61 | 5.52 | 7.62 | 5.69 | 7.66 | 5.52 | 8.15 | 5.58 |
| A₁₀₊ | 25.47 | 1.93 | 25.67 | 1.09 | 25.63 | 0.76 | 25.97 | 0.57 | 22.29 | 0.65 |

| | | | | | |
|---|---|---|---|---|---|
| Reactor pressure (psig) | 200 | 200 | 200 | 200 | 200 |
| Reactor temperature (° F.) | 700 | 725 | 750 | 775 | 800 |
| Benzene/Xylenes | 0.50 | 0.56 | 0.60 | 0.65 | 0.63 |
| Toluene Conversion | 26.72 | 25.40 | 26.28 | 26.27 | 30.00 |
| EB selectivity in C₈ fraction | 14.36 | 7.96 | 3.98 | 1.89 | 1.18 |
| C₉₊ aromatics conversion | 70.59 | 78.30 | 81.51 | 83.96 | 82.85 |

Based on the foregoing data, at constant pressure and WHSV, the conversion of C₉₊ aromatics increases as the temperature increases. Similarly, at constant pressure and WHSV, ethylbenzene selectivity in the C₈ aromatics fraction decreases as the temperature increases. Toluene conversion, however, does not significantly change in response to temperature changes when the pressure and WHSV remain constant.

The foregoing description is given for clearness of understanding only, and no unnecessary limitations should be understood therefrom, as modifications within the scope of the invention may be apparent to those having ordinary skill in the art.

What is claimed is:

1. A method of making xylene isomers, the method comprising contacting a feed comprising C₉ aromatics with a non-sulfided catalyst under conditions suitable for converting the feed to a product comprising xylene isomers, the catalyst comprising a support impregnated with a hydrogenation component, the support comprising a macroporous binder and a large pore sieve.

2. The method of claim 1, wherein the hydrogenation component is a metal or oxide thereof.

3. The method of claim 2, wherein the metal is selected from the group consisting of Group VIB metals, Group VIIB metals, Group VIII metals, and combinations thereof.

4. The method of claim 3, wherein the metal is a Group VIB metal.

5. The method of claim 1, wherein the catalyst has a macropore volume of about 0.02 cubic centimeters per gram (cc/g) to about 0.5 cc/g.

6. The method of claim 5, wherein the catalyst has a macropore volume of about 0.1 cc/g to about 0.35 cc/g.

7. The method of claim 6, wherein the catalyst has a macropore volume of about 0.13 cc/g to about 0.3 cc/g.

8. The method of claim 1, wherein the sieve is selected from the group consisting of large pore zeolites, large pore aluminophosphates, large pore silicoaluminophosphates, and mixtures thereof

9. The method of claim 8, wherein the lame pore zeolite is selected from the group consisting of mordenite, beta-zeolite, Y-zeolite, and mixtures thereof.

10. The method of claim 1, wherein the catalyst comprises about 0.1 wt. % to about 20 wt. % of the hydrogenation component, based on the total weight of the catalyst.

11. The method of claim 4, wherein the Group VIB metal oxide is molybdenum oxide.

12. The method of claim 11, wherein catalyst comprises about 0.5 wt. % to about 10 wt. % molybdenum oxide, based on the total weight of the catalyst.

13. The method of claim 1, wherein the macroporous binder is selected from the group consisting of aluminas, aluminum phosphates, clays, silica-aluminas, silicas, silicates, titanias, zirconias, and mixtures thereof.

14. The method of claim 1, wherein the support comprises up to about 50 wt. % binder, based on the total weight of the support.

15. The method of claim 1, wherein the support comprises about 10 wt. % to about 30 wt. % binder, based on the total weight of the support.

16. The method of claim 1, wherein the support comprises a sieve:binder weight ratio of about 20:1 to about 1:10.

17. The method of claim 1, wherein the feed is substantially free of ethylbenzene, xylene isomers, and sulfur.

18. The method of claim 1, wherein the feed is substantially free of non-aromatics.

19. The method of claim 1, wherein the feed comprises less than about 50 wt. % toluene, based on the total weight of the feed.

20. The method of claim 19, wherein the feed is substantially free of toluene.

21. The method of claim 1, wherein the feed comprises less than about 30 wt. % benzene, based on the total weight of the feed.

22. The method of claim 21, wherein the feed is substantially free of benzene.

23. The method of claim 1, wherein the feed is substantially free of C₁₀₊ aromatics.

24. The method of claim 1, wherein the conditions comprise the presence of a hydrogen-comprising gas.

25. The method of claim 24, wherein the hydrogen-comprising gas is present in a molar ratio relative to hydrocarbons in the feed of about 0.1:1 to about 10:1.

26. The method of claim 1, wherein the conditions comprise a temperature of about 200° C. to about 1000° C.

27. The method of claim 1, wherein the conditions comprise a pressure of about 0.3 MPa to about 15 MPa.

28. The method of claim 1, wherein the conditions comprise a WHSV of about 0.1 to about 20.

29. The method of claim 1, further comprising separating at least a portion of the xylene isomers from the product.

30. The method of claim 29, further comprising recycling to the feed a portion of the xylene isomers-lean product.

31. A method of making xylene isomers, the method comprising contacting a feed comprising C₆-C₈ aromatics and substantially free of C₉₊ aromatics with a non-sulfided catalyst under conditions suitable for converting the feed to a product comprising xylene isomers, the catalyst comprising a support impregnated with a hydrogenation component, the support comprising a macroporous binder and a sieve selected from the group consisting of a medium pore sieve, a large pore sieve, and mixtures thereof.

32. The method of claim 31, wherein the hydrogenation component is a metal or oxide thereof.

33. The method of claim 32, wherein the metal is selected from the group consisting of Group VIB metals, Group VIIB metals, Group VIII metals, and combinations thereof.

34. The method of claim 33, wherein the metal is a Group VIB metal.

35. The method of claim 31, wherein the catalyst has a macropore volume of about 0.02 cc/g to about 0.5 cc/g.

36. The method of claim 35, wherein the catalyst has a macropore volume of about 0.1 cc/g to about 0.35 cc/g.

37. The method of claim 36, wherein the catalyst has a macropore volume of about 0.13 cc/g to about 0.3 cc/g.

38. The method of claim 31, wherein the medium pore sieve is selected from the group consisting of medium pore zeolites, medium pore aluminophosphates, medium pore silicoaluminophosphates, and mixtures thereof.

39. The method of claim 38, wherein the medium pore zeolite is selected from the group consisting of aluminophosphate-eleven (AEL), Edinburgh University-one (EUO), ferrierite (FER), Mobil-eleven (MEL), Mobil-fifty seven (MFS), Mobil-Five (MFI), Mobil-twenty three (MTT), new-eighty seven (NES), theta-one (TON), and mixtures thereof.

40. The method of claim 39, wherein the Mobil-Five (MFI) zeolite is selected from the group consisting of ZSM-5, silicalite-1, related isotypic structures thereof, and mixtures thereof.

41. The method of claim 39, wherein the Mobil-eleven (MEL) is ZSM-11, related isotypic structures thereof, and mixtures thereof.

42. The method of claim 31, wherein the catalyst comprises about 0.1 wt. % to about 20 wt. % of the hydrogenation component, based on the total weight of the catalyst.

43. The method of claim 34, wherein the Group VIB metal oxide is molybdenum oxide.

44. The method of claim 43, wherein catalyst comprises about 0.5 wt. % to about 10 wt. % molybdenum oxide, based on the total weight of the catalyst.

45. The method of claim 31, wherein the macroporous binder is selected from the group consisting of aluminas, aluminum phosphates, clays, silica-aluminas, silicas, silicates, titanias, zirconias, and mixtures thereof.

46. The method of claim 31, wherein the support comprises up to about 50 wt. % binder, based on the total weight of the support.

47. The method of claim 31, wherein the support comprises about 10 wt. % to about 30 wt. % binder, based on the total weight of the support.

48. The method of claim 31, wherein the support comprises a sieve:binder weight ratio of about 20:1 to about 1:10.

49. The method of claim 31, wherein the feed is substantially free of ethylbenzene, xylene isomers, and sulfur.

50. The method of claim 31, wherein the feed is substantially free of non-aromatics.

51. The method of claim 31, wherein the feed comprises less than about 30 wt. % benzene, based on the total weight of the feed.

52. The method of claim 51, wherein the feed is substantially free of benzene.

53. The method of claim 31, wherein the feed is substantially free of C₁₀₊ aromatics.

54. The method of claim 31, wherein the conditions comprise the presence of a hydrogen-comprising gas.

55. The method of claim 54, wherein the hydrogen-comprising gas is present in a molar ratio relative to hydrocarbons in the feed of about 0.1:1 to about 10:1.

56. The method of claim 31, wherein the conditions comprise a temperature of about 200° C. to about 1000° C.

57. The method of claim 31, wherein the conditions comprise a pressure of about 0.3 MPa to about 15 MPa.

58. The method of claim 31, wherein the conditions comprise a WHSV of about 0.1 to about 20.

59. The method of claim 31, wherein comprising separating at least a portion of the xylene isomers from the product.

60. The method of claim 59, further comprising recycling to the feed a portion of the xylene isomers-lean product.
